SEQUENCE LISTING (14 sequences)

SEQ ID NO: 1 - DNA, 1001 bp, Homo sapiens
cctgcggcccggtgtgggatccacagagtgaagccagctgggctccagagtctggtggggacttggagaacctttatgtctagctaggggattgtaaatacaccagtcagcactctgtatctagctcaaggtttgtaaatgcaccaatcagcactctgtgtctagctcagggtttgtaaatacaccaatggacactctgtagctagctaatctagtggggacatggagaacttttgtgtctagctcagggattttaaacgcaccaatcagcaccctgtcaaaacggaccaatcagctctctgtaaaacagaccaatcggctctcggtaaaatggaccaatcagcaggatgtgggtggggccagataagaaaataaaagcaggctgcccaagccagtagtggcaacacgttcaggttgtattttacagtgtggaagttttttgttttgttttgtttttgttgttattgttttctttctctttgcaatgcgttaaatgctccttactctttggatgtacactgcttatattgaggtgcaacattcaacatgagggtttgcagcttcactcctaagccagggtagatcacgaacccaccagaaggaagaaactctgaacacatctgaacatcagaagaaacaaactgcgaacgggacatgctgcgtttaagaactgtaacaccgcgagggtccgtggcttcattcttgaagtgagtgagaccaagaacccaattctgaatacactgggattacaggcatgaaccacagtggctggctggcagcagtaagtttaacctgaacctcgagcttctttgatgtgctgtgatgcagtacaatagatcagcacacacatcatatattcataaatacacactaggccttagtgtgtcttctcaaccctcagagacgtttaagtgacttgctcagggaaatgctaccaattgcaaatatggtggtgattcttaatcattactatatctgaaagtgaaaagtcaatggtttaa

SEQ ID NO: 2 - DNA, 1001 bp, Homo sapiens
aaagtggaaggggcccttgagtgttgagatctgggtcctagaccctgctcagataaggcatctggacaaccttggacaaggcactgaactttcatggacaatgtgtatatcatctatgtaaaatgcaagctctgaatacacgacttctaaactcccttcaaacactcacatttcattaacaataaagtcattgttcactaagagaacattctgaagccatctggaattctgcctctttcctctgccttcttgtacctgtgaagggagtggaaggaacccatggtgcccacgtcgagtgtggaaagaagcctgagatgtttaggccacggaatggacatcatttcagttgaaactttgggcaatgcactaccagggtcacagccttttcctatagtgtttctgggacctgtccgctctgaaatcagtgtgcttcttctcccccactgtccttttgtgactactggtcttcatggctatgtttggaagcaagaacatctctatcaggtcttgtttaacttgagccttgtcccggctggctgagatggtgcgagctagggaagccagtgggaagggcgggaatcatctatgggaagttggccaaaatcttactaaggaactgggattcctaaatccctcactgggagaatttattccagtattattccatgggctgggcgtggtggctcatgcctataatctcagcattttgggaggccaaggcgggaggactgcttgaggccagcagttcaagaccagcctgggcaacatagtgagaccccatttctaaaaaaaaaaaaatacaaaactaaacttagctgggtgtggtgggcgagggcctgtagtcccagctactcaggaggctgcagtagaaggatcatttgagcccaggagttggaggctgcagtgagctgtgatcatgccactgtactccaacatgggtgacagagtaagcccctgtctcaaaaaaagaaaaaaaaaaaaaaggacaggacatccatgtt

SEQ ID NO: 3 - DNA, 1001 bp, Homo sapiens
ccttggtgagacagagagaaagagagagagagagatcttgtgtctcttcctctttcataaggacacaatcctatcatggtgctccattctcatgacctaatctgatcctaattagctcccaaggtcccatctccaagtaccattacactggggattagagcttcaacatacgatttggggaaggacgctaacctactgtctacaacaaagctctactcactgaaatcacctacatttgcttctcctagaactcatcttgctgggcattgcttccaccttcaatttttgttaccaaactggagcactgtatccaggacctgaaatctgtgaactcaagggtttttggctttagcctcagccattatttttctattggttgtattctgatataatgtgaccccagattcagaaagtaggaagacaagttcttgctttgggggctgaaatgcaaactgaaggtgaaggaaggacaatgaatgctcagttggcatttcttaggtttcaagcaaagaagaagagatacaggtggggaagcaagtgatgatgtagcaaccagattatgtagttgttgatactcactgctttatttcccccactggccctccatagctttaccagagtcttccctaaacctggctggcagctgattagaaatgccaaatttctaatgccactccaggcctgctggatcagaacctacaatatatgacaaagctggcgggtcattcacaacaactttacagttagagaaacaccgctctgaaagtctgagtgccaggtactctgtggttcagggtgtggggagagatttgagatcctacacttggctataatgttaatgggacataattatttactaagctcctcccacaagcaaagtgaatttcaaggaagaaatgttgggcttacatgaacgtctcttctctttatggaaagttacttttcattgctaatattggaagactttttttttgtaagaaagttatattttctttcctgta

SEQ ID NO: 4 - DNA, 1001 bp, Homo sapiens
tgagtgtatgtcataattttctcagagggtgtctacatctcaatgaaaggaactattctttttcactgtcaggaaaatgtgaaactctagaaagcatcatgcagtgtgaaactaaagagggctgatatttacctaaaaactgttgatttaagctgcctgatgatagatagctccccgaaacgaaaaactactgtgaatttttttggtctttgcccccatcagaggttgacattgtcattttccaatgaaataagtaacaaggacttcaggatcttgtttggtggccaaagccactgtagaaaactaatcagagtctctcattctgtaagtaaatctctctgcggattaaaaggctctgttctgaatctcaaacacataaatggaagctcttatgatattgccagagcagtggtctaatatacatattatggcaggcaactttaagacttaatcatctttccagagagccatgtagttactggctcaggtcaggggagaaaagatcatcataggagcctgaaaaagaaccttagctctttaatagtttgtaccttgtgattgcccagcaaggcagtatttgaagattcttcttttaatcctttttggagttctcatggaaagatgcttttagcagtctcatgcatgtctatttctaagaaagtcacacttggtgatctgtagcacatttactatataagcttaacatgactggtactaatgcaaatcccatgtggattatatctgaaccttgtgccttttcagagtcacagaaaatagactatcactcaaaccataggaaatttgggaggctgtaagataatggtgactgaaccatactacatttcccctgctcagatgtggctgtctcaaaaactacttatacccttaacattaaactcagcgtctttaaattatcactgctcctggccccttattcattggtgtccacatttcttcttctaggcagaaatactctcttttaatttggatgctctgagtgg

SEQ ID NO: 5 - DNA, 1001 bp, Homo sapiens
acatatagtttagttgcatcagtcagctttatatttaaagtttccgagcttcttctacttctgatgaaagtctggtgagcaaagatactcttcatagacaggaacattttcctggaatagctcaggcccaggaaaaaaaaatttcccagaggccccttgtcaagagttagttttttaattgcctggggctccatttatcctcttatcttctaaaaattcttcccaattttcccagccatatttaagtcccgctaactccatgaaatcttcccacatcttctccaatccacagatttcttcttttgattaattctcacaaatgccatattctaggaaacattgttggagcatttaccctttctctattaatattagttttgctggactgtaagtgcagcaaaaacagtgacggtttatgattttctgtcgacatccattacacgacctagaatcatctcaagtgtatacacaggactcagtaaatatttgtgggttctaatctcaagtcaataagaacaacaaaaatccctacttgtaaaaaaaaatgcccattttttctttttctttcttaacccaataatgaaatccttgggaaaatatgtttacataggaatcttaatattgccatgtcagtttgagaaaaggatattctttatttggtgtgtcactggatttgtgacagttcttatgaaccatgacttgaaatgagataggttagtaaacatttgcgagtttttctatggtctctaaacagagccctttgaaaagagcaaactcagattctggcaaactttagataacttcagacagagagagaaaatctctgtccaaagcagtatttaccaaatttatttggagttatttgtaaaagaaaaggttttgatggacacagaaggcttgagaaatcatgattaaacaaagttatttgtgtttcttcatcacaaaatttctcagtacttttaatatgctaataacttctaagaaggaaagcatttcccaaa

SEQ ID NO: 6 - DNA, 1001 bp, Homo sapiens
aatatattcttgttagtattaaaatcttactaaatatcagcctttcttctggtaattacaaaagaataaattctttctaagtaactttttgaatacaggttggataaaatttggaatttatcttagaatattttctgataatctagtaatgtgaagctctctagttatttcttttctgcctctttgaagttacacttgaatgctcttttcctggaactgatatttttctttacctttctaaataagatgaaaagaacagacagtgcttttctcagaggtggttatatttatgatttccaattccagcgtattagatattcccataccagaaggtactgtgacacttgtccagtttttattatatcaaactaagttcccaaactgattgtgtttccttatttgtaaaagcattactcacttaggggaacaaacttcctaggcaataaggaagaaaatatacatttcatgtttcatttcacttattcagtaacatattaaaccttgattacaaacattcattttttggactcatattgcatcgcttaatgccaacttaggaaaaaatgggcaatcttaatcataccttttggcatactaaatagcacttggacaatatatagcaaaatagggtaacaaatttcaaatttgttacaattattataatggcttcttttagggctaataatccatcctgtttctcagcaaattaaataattaatcaaagagactatcacatatttaagtcactatacatgtaatacttacagaaatatattttctatgtatatgtgtaaaattacaagatttcaaactgaaacctaaaataatttgaatcattacagaattaaatataccttaaaggaaaagggtaattcaaaagtggtgcccaaatctatgtctgatattaaaacataacattttcttcctatgacacaatataagatatagcaaaaaatatagtataagtcatcccaaattataccaaaccttcctttcaagga

SEQ ID NO: 7 - DNA, 1001 bp, Homo sapiens
gaatatcttatatataaaatatacaaaacctcagttttataaaattatcataagccctccatacttttacaaatccttctcggtcttcagattcagaaatggtgtatctataataggaactaattctgaaagcacttaaaaagaacaatgtcaggacagctaaatatatgacttataaaaatcactagttcctcccaccttggttctgttgaccttagtggccaagtccttttcctgagcttttaataaacacatgcctgtaagcaaagatacgcatagttaaatctatacttagcatattaacccccagatgagcataaagacatatgctaccagagatccaggaaacaagaatctgggttttggggctttctgtattgggggtgaatgttcaccttcccacaagagtctcacaatggtggctttcccagccctctaggaaggggcttcctcaaaaaaagtagcatctccaaacactcatacattgtgaagcaactcaagtttccaagtaaaaagatattaaatgtattaatagaaaggactgcagactacccttaaaatatatatacgtgtatatatatatatatttttatgtatatgtgtgtatatatatatatatttatgtatatgtgtgtatatatatatttatatatgtgtgtgtatatatatttatatatatgtgtatatatatttatatatatgtgtatatatatttatatatgtgtgtatatatatttatatatatatgtgtgtatatatatatttatatatatatatatatatatatatatatatatatattcccagctctttgggaggctgaggtggaaggatcacctaggtcaggagttcatgaccagcctggccaacatggtaaaaccccatctctactaaaaatacaaaaaaattagccaggcatggtggcatgtgcctgtaatcccagctacttgggaggctgaggcaggagaatcacttgaacctgggagggggaggctgcagtt

SEQ ID NO: 8 - DNA, 1001 bp, Homo sapiens
accaacatggagaaaccccatctctactaaaaatacaaaagtagctgggtgtggtgacgggtgcctataatcccagctagttgggaggctgaggcaggagaatcccttgaacccaggaggcagaggttgcagtgggccgatatcgcaccattgcactccagcctgggctacgagagcaatactccgtctcaaaaaataaaagaaaaaaaaaaagaaaaggagacagaataggagaggaaagaagacagaaggaagacaggatggggtgggggccctgggatgatgagaggaggcatagagacagagaaaggggagaggaggagaggacaggaacaaagaaaggctgggactggaaagcataggaggagggaagggcgagaacaggcagggacaaccaagcagggaggtggaagcaaagaggttgggccagccatgggatctctagcaggctcttcctttgatgaaaatgcacattagcaaacgtgctaggatctgacaagtgccaacaggcccatcgcagagttaaaaagctctacatctgtcagggtttcaatttttaagtcagcactttccttcttctcatgctgggattctgcaactgtggtttccccccacccccatgtctcccgccagtgcgtggggcttgtcatggagccgtcagacttccgaggagcctggcggggcagagtgggtatgggttgggggggtggtctatgcagggtgagggtggtggcctcctccacagcagctgttgcagggatgtcctgtcgaaggagggcccagggaagtgggtgatgtggtaatgttctcagcagacactgggagtctccaaggcaatgaacttctgtaatgggttgaactgtgtccccccaaattcatgtccacgcagaatgctgggatgtgacattatttggaaaaagggtctttgtaggttgatatggtttggctgtgtctccacccaattctcatcttgatttgtagctcccataactcccaagtgt

SEQ ID NO: 9 - DNA, 1001 bp, Homo sapiens
gcctgggtgacagagtgagactctgtctcaaaaaaataaataaataaaaataaagcaacactgtctaatagaaatatgtgatccacatataaaattaaaacattttggtggccacattaacaacaaaaacaggtgaaattaattttaataattttatttaatccaatgtatcaaaaatattaatgaattttacatttttggtatgaaatccttgaattcagtgtgtatttgatgactatagtacatctgaacttaaactggccacatattacatgttcaacagccacatgtggctagtggctaccatactggacagcacagatttacagatctgatgtaatcctaatcaaaattgaagcagatatttttgtggaaatcaacgtgctgattcttgaaaaataacaatactagaaaatataaccaccacatattgagacttattataaaactatacgaagagaatgtggcatcggttcaaggatagaaatagactgacggaaaagaatagggtcaggaaacacatgtgtggtcacctgattatgataaagcaggacaccagggaaagcatgctgatttcattaaattgtgccgagtcagtgccttacaggaacaaaaaaaaagatcttgactcctaatgcattacattacatacatacaaaaatcagttctagatggattgtagagttaaatgataaaaaacctttcagaggaaaacaggaaaaaaaatcttcacaccattgggaataggcaaagatgacttaaaccggacacacacaaaaaaacacggataaatgagactaaaaatgatttgcttacttcttccggatgaaaggtaggaggcaaggttggtgaaggtgcaaaaggaggtggagagataacctttctttcctctagctgggccattatttcctttcgtcggcgatgtagttcatctagactaggatgaggctggggaggaggaggaagccggctataaggaggttcctaaaaatagaaagatt

SEQ ID NO: 10 - DNA, 1001 bp, Homo sapiens
acagcacactcaccaccattgaaagcaggggatatacttgtcaacctcctcgctcttgctcttgttgatcctcttcctgttataccctttcatccaattcttttcagtcttggagtctggctcaaatctcactatttttgtgaagctttctctaatctccacccatcatccttcttcccctcccctcccaagttggcagctatatctcttcctctatgcatccacaagcatttataaaaaaagtttcattgtataacatctgatatggcatattggcatattctaccactattccacatgccaaatcttaaattgtgttatatttatttgtatgaatttcttttccctcctagttggtaggagtttccttgagatcatgtctacttattctgaatcctggagtgtctaccagagttcaccactcaaacagctattatattgtggtgttttgtgtttttcactagactgcaagggcctaatgaggaagacatgttctctaactttgtatcactattacctaatcacaggccaagcaggtcctgtggcagagcacgtcatgagaaactacttgccttggtgctgaccattaagtcagtgtccaaattcacagtttccttgtgagtttatgaagtttccatagcagcttcctgggctatagaaaccacatatcccatttattgtctcttctactcttggtaaactctccttttatcagtgaaggggaggagcatacatatataacctgcccacccacacctaccactcctcctcaccaaaatcccccaccctctgaatcaaatggtcgtattctgctttgaataaattaattctaatttcctggacatttttagattattcatctacagggatatgtacagggaccatgttgatgttaatttatcactcattttaaccaggatgtctaagaacaggtaagactaaatctcatgaaacatttaaaaacagtgaaaccagtaagtaatatattaccaaaatgagtt

SEQ ID NO: 11 - DNA, 1001 bp, Homo sapiens
tttcaaaggtgtagagttccatagtgttaagtatattcacattattttgtaaccaatctctggaatgttttcatcttgcaaaaataaaactatacctattaaacaactatacctattaagcaactgctcccttttccccaccccaggcctggcaaccaccattctactttctatccatctgattagtctagcacagtgtcctccaggttcatccattgttttcactgttagaattggggctttcttatgacttttctggttttgtttttgcccacatactttatctgagaaaataattttatacttaaattaagtttctggtagtggctattctcagcaattatttatggtgacactctatttggtaagctcagtatagtatagttttcctatttctctctctgtcttttttcttatttacggtatttgtttgcaatgataaaatggcttttctaagaaacaaacattccatttaaggcactatcacctcaaatcctcttttagctagacaatgaaagaaattgggataaaaatacatttcaaaagtaaaattaaatatagtccctttattccatggtgcatatgtgccacattttctttatccagtctaacattgatgggcatttgggttggttccaagtctttgctattgtgaatagtgctgcaataaacaaacatgtgcatgtgtctttatagtagaatgatttataatcctttgggtatatacttaataatgggatgactgggtcaagtggtatttctagttctagatccttgaggaattgccccactgtcttccacaatggttgaactaatttacactcccaccaacagtgtaaaagtgttcctatttctccacatcctctccagcatctgttgtttcttgactttttaatgatcgccattctaactggtatgagatggtatctcattgtggttttgatttgcatttctctaacgaccagtgatgatgagcttttttttcatgtttgttggccaca

SEQ ID NO: 12 - DNA, 1001 bp, Homo sapiens
atttactaaatttgatttggtacaacaactgaataagtcataccaattcttggctctgtgttccaaacagagtaaaaaattgctggtaatgcagttctcaactctcaacaggcagtttccatctatttgacatcatttgaatccaagctaaaagtagaaatggcataagtagttgaaagtcaaagtacacagctttattctctgctgctataatatagtttggaaatgtatccgtgaaggcactaaagtcaaaaaataagctcatattgaaaacatagatttttaaaaataccaccgtcaactgtttatctatatgaagtggttttcacaggcttttcccttgaaaaatgtcactgcacctgttaaacaagtgttcaaagagatttgctatgaattattccatgaaatgtcctatgttttagacaggttcctttctttggtgggggttggggggctgtggtatacaaacctaccttgactgattatattaaccacaaactataacaacaaacaaaacaaatcttttgatgtttgcagattcgggatggttgctcagctaacaagtatttctctagtagccatgtctagggaactcccttctttctgggaattgtattcccttcctcccagatttagacacatgtcccagatgggaactgtcctttcctcatgactgcatttcccccgggatgaggtaggaaccttactctacctagttcaatgagcttaatcctaggactctccaataatcacatctccacacaccatccctgatcttcactgaccttcagtgactgttttagataagagtttaagagacaagttagccaattaaatgtccttcccaggtttttaaccctttgttagaggaagataatctctcttctcctgtctaaatactgtgacgttgtgaacctggagatgctggaagctgcatttgtactcttgcagcaatgttattcagagataatgaagcaaatgtgaaagagaaagaatgctca

SEQ ID NO: 13 - DNA, 1001 bp, Homo sapiens
ttagagtttgagggtctggatcatcattcaatgtcagtttgagataatgacttcttggtctgtgcatataaacattgttgcagggatgaggctaatgatgaaggcacatgtgatagatatttttacataatttgggtatgaacttttttgcaggtgttggttaaggtagcagtaattggtaggcaaggggactagggctgttgcatcagtggaagaatacatgcttgttacttttatttggagttgcaccagtgtttttggttcttaagaccaacggatgactctcatcctttaaaagttgagaaagccatgttgttagacatgggggcatgagttagcagttcctgcatacattctcggtagataagaaattgaaggcttctattgttagatccacaatctaatgttttgcttaaactacagctacagcatgcaaaccccaaaataattttagggtctaaggatagtaggaagataggtgctagatatataagtattaatgtgtttttctcgtgtaaaggaaggtttgatatgttgatatgatacgtaagtgtccttcgttgtgattagcatacacagggagtaaagggctgtaattagtatactaagtcctataagcataatagtgatatttgacgaggagagtgaggccatagtcacaaagagttcttctactagattaatggtagggggtaaggcaaggttagtctgatttgctagaagtcatcctgaggctattagtgggagtagtgtttgaaggcctcagtaagtaatatggttcatctatgggctcactcatagtttgagtttgctaggcagaatagtagggatgaagtgagtccataagcaattataggggtgaccgcacctgtaaagctccaagggatctggatgagaatagccataataacaagtgctatgtggcttatggaggagtaggcaataagtgattttaaattggtttgttgtagacaaatagagcttgtcatgattattcctca

SEQ ID NO: 14 - DNA, 1001 bp, Homo sapiens
ctctacctcttttctagttttcactatatcttgcaaattaacaacttgcccttgaatctttgttagagtctttttctgaggaactcaaaccaagatataattcacgtaaaggatttagcacatagcaaacactaaaaaatacaacaaagcaaacactatgttattactattggataacctcttacttttcctgtttcatctcaaaagttctggattgttgaatatcatgaagcaatgcactgcccttttgaatgcttaatctttttttccaagctcttaattgtcatggtatacataagaaaaaaaattatttgagataaaatcatagcgcctgtgagaggttgttccccaagccacagtttttgttacataatgttttccctgtggatctctggatatttcagtgagtaaccaatgaactccatcctatttatatatctgtataaatttagctgtttacaggatgtttgcctgattaatgaatggagaaggatgaataacggaaatataccaataataatatgataatgacatcaaagtatacttgtgttatgacatgaatattaaaggtcaggcagttctaggaattctaaattcatcttaaaggacatttcagtctttaactaatggaaaggtgaacttcaaacatactgaccttgggtttttgttttgactattagttttgatgtggatgcatttgaaattcacaggaacagaggttcttctttgtcattgattagtgcttctccaagtgaagactcttttggatggattttatttgtgttttcttcttgaaagaaatataaagaaataagcatcatgcattagggtatttgaaatataagacttgtgacgcttgtctatatattttatttaacaaattggttaagattatgggctctggagtcaggcagtctgagatcaaattgtggttctacaactagcaaaatacttgattttggtcaagttgctccatgtgtctactaagccttcaatctaaa

(19) United States
(12) Patent Application Publication
(10) Pub. No.: US 2014/0148344 A1
(43) Pub. Date: May 29, 2014

(54) ASSOCIATION MARKERS FOR BETA THALASSEMIA TRAIT

(71) Applicants: Sina Vivekanandan Thrissur Kadavil, Thrissur (IN); Sunil Kumar, Bangalore (IN); Randeep Singh, Bangalore (IN); Nevenka Dimitrova, Eindhoven (NL)

(72) Inventors: Sina Vivekanandan Thrissur Kadavil, Thrissur (IN); Sunil Kumar, Bangalore (IN); Randeep Singh, Bangalore (IN); Nevenka Dimitrova, Eindhoven (NL)

(73) Assignee: KONINKLIJKE PHILIPS N.V., EINDHOVEN (NL)

(21) Appl. No.: 14/007,673

(22) PCT Filed: Mar. 29, 2012
(86) PCT No.: PCT/IB2012/051520

§ 371 Date: Jan. 7, 2014

Related U.S. Application Data
(60) Provisional application No. 61/472,228, filed on Apr. 6, 2011.

(51) Int. Cl. C12Q 1/68

(52) U.S. Cl. CPC C12Q 1/6883
USPC 506/2; 536/23.5; 435/6.12; 435/6.11

(57) ABSTRACT

The present invention relates to isolated nucleic acid molecules of SEQ ID NO: 1 to SEQ ID NO: 14 which show a single polymorphic change at position 501, where the wildtype nucleotide is replaced by an indicator nucleotide, respectively. The present invention further relates to the mentioned nucleic acid molecules wherein a panel of 1, 2, 3, 4, 5, 6, 7, 8, 9, 10, 11, 12, 13 or 14 of the polymorphic, changed sequences comprising the mentioned indicator nucleotides constitutes a marker for beta thalassemia, in particular of beta thalassemia minor. Further envisaged are specific panels comprising SEQ ID NO: 1; or SEQ ID NO 1 and 2; or SEQ ID NO: 1, 2 and 3, or SEQ ID NO: 1, 2, 3 and 4; or SEQ ID NO: 1 to 5; or SEQ ID NO: 1 to 6; or SEQ ID NO: 1 to 7; or SEQ ID NO: 1 to 14; or SEQ ID NO: 8 and 14; or SEQ ID NO: 8 and 9; or SEQ ID NO: 2, 4 and 13. The present invention further relates to a method of detecting or diagnosing beta thalassemia, preferably of beta thalassemia minor, in a subject, comprising the steps of: (a) isolating a nucleic acid from a subject's sample, (b) determining the nucleotide sequence and/or molecular structure present at one or more of the mentioned polymorphic sites, wherein the presence of an indicator nucleotide indicative of the presence of beta thalassemia. Also envisaged are a corresponding composition for detecting or diagnosing beta thalassemia, the use of the mentioned nucleic acid molecules for detecting or diagnosing beta thalassemia or for screening a population for the presence of beta thalassemia, as well as a corresponding kit. The methods, compositions, uses and kits of the invention also relate to the assessment of the risk of developing beta thalassemia in a subject and/or in a subject's progeny.

### FIELD OF THE INVENTION

The present invention relates to isolated nucleic acid molecules of SEQ ID NO: 1 to SEQ ID NO: 14 which show a single polymorphic change at position 501, where the wildtype nucleotide is replaced by an indicator nucleotide, respectively. The present invention further relates to the mentioned nucleic acid molecules wherein a panel of 1, 2, 3, 4, 5, 6, 7, 8, 9, 10, 11, 12, 13 or 14 of the polymorphic, changed sequences comprising the mentioned indicator nucleotides constitutes a marker for beta thalassemia, in particular of beta thalassemia minor. Further envisaged are specific panels comprising SEQ ID NO: 1; or SEQ ID NO 1 and 2; or SEQ ID NO: 1, 2 and 3, or SEQ ID NO: 1, 2, 3 and 4; or SEQ ID NO: 1 to 5; or SEQ ID NO: 1 to 6; or SEQ ID NO: 1 to 7; or SEQ ID NO: 1 to 14; or SEQ ID NO: 8 and 14; or SEQ ID NO: 8 and 9; or SEQ ID NO: 2, 4 and 13. The present invention further relates to a method of detecting or diagnosing beta thalassemia, preferably of beta thalassemia minor, in a subject, comprising the steps of: (a) isolating a nucleic acid from a subject's sample, (b) determining the nucleotide sequence and/or molecular structure present at one or more of the mentioned polymorphic sites, wherein the presence of an indicator nucleotide indicative of the presence of beta thalassemia. Also envisaged are a corresponding composition for detecting or diagnosing beta thalassemia, the use of the mentioned nucleic acid molecules for detecting or diagnosing beta thalassemia or for screening a population for the presence of beta thalassemia, as well as a corresponding kit. The methods, compositions, uses and kits of the invention also relate to the assessment of the risk of developing beta thalassemia in a subject and/or in a subject's progeny.

### BACKGROUND OF THE INVENTION

Thalassemia is an inherited genetic, i.e. autosomal recessive blood disorder. The genetic defect, which can be a mutation or a deletion, typically results in a reduced rate of synthesis of one of the globin chains of hemoglobin, or in no synthesis of these chains. As a result, abnormal hemoglobin molecules are formed, which lead to anemia, i.e. the characteristic symptom of all thalassemia forms. In typical cases thalassemias are thus related to quantitative problems of a reduced number of globins synthesized, often via mutations or modifications in regulatory genes or regions, whereas the other predominant anemic disorder sickle-cell anemia is caused by the qualitative problem of the synthesis of mal-functioning globins. Thalassemias are categorized in two main forms, alpha thalassemia and beta thalassemia according to the chain of the hemoglobin gene which is affected: in alpha thalassemia the production of the alpha globin chain is affected, whereas in beta thalassemia the production of the beta globin chain is affected.

In the etiology of alpha thalassemia at least four alleles of the genes HBA1 (encoding hemoglobin subunit alpha 1; located on chromosome 16 p13.3) and HBA2 (encoding hemoglobin subunit alpha 2; located on chromosome 16 p 13.3.) are involved, as well as a deletion of chromosome 16p. This results in a decreased alpha-globin production, and a concomitant excess of beta-globin chains in adults, which form unstable beta globin tetramers, also called hemoglobin H, showing abnormal oxygen dissociation curves. Normal hemoglobin is, in contrast thereto, provided in the form of a heterotetramer of two alpha and two beta subunits, also called hemoglobin A.

In the etiology of beta thalassemia in principle mutations of the HBB gene (encoding hemoglobin subunit beta; located on chromosome 11 p15.5) or of associated regions are involved. Up to now more than 470 mutations associated with the HBB gene have been recorded in HGMD and other databases, which may lead or contribute to beta thalassemia. These mutations include small point mutations or reading frame shifts within the beta globin locus, as well as a few larger deletions in said region. The mutations may, for example, have influence on the correct splicing of primary beta globin transcripts and lead to aberrant splicing pattern. A different type of mutations may occur in the promoter regions preceding the beta-globin genes. In all cases, the absolute or relative absence of beta chains leads to an excess of alpha chains, which, however, do not form tetramers, but bind to red blood cell membranes, produce membrane damage and even may form toxic aggregates.

The severity of the disease apparently depends on the nature of the mutation. In beta thalassemia major or Cooley's anemia any formation of beta chains is prevented. In particular, the disease may occur if both alleles have thalassemia mutations. This typically leads to a severe microcytic, hypochromic anemia. If not treated it will cause anemia, splenomegaly, and severe bone deformities. It normally progresses to death before the age of twenty. Treatment typically consists of periodic blood transfusion, splenectomy if splenomegaly is present, and the treatment of transfusion-caused iron overload. The genetic situation, or the mutations leading to it, are typically described as β⁺/β⁰, β⁰/β⁰, or β⁺/β⁺, wherein “β” describes alleles without a mutation that reduces the function of beta hemoglobin, “β⁺” describes alleles comprising mutations which allow some beta chain formation to occur and “β⁰” describes alleles comprising mutations which entirely prevent the production of beta chains.

In beta thalassemia intermedia, some beta chain production occurs. Affected individuals can often manage a normal life but may need occasional transfusions e.g. at times of illness or pregnancy, depending on the severity of their anemia. The genetic situation or the mutations leading to it, are typically described as β⁺/β⁺ or β⁺/β⁰.

In beta thalassemia minor or beta thalassemia trait only one β globin allele bears a mutation. This is considered a mild microcytic anemia. Thalassemia minor is not life threatening on its own, but can affect the quality of life due to the effects of a mild to moderate anemia. It is not always actively treated and may even be unnoticed, in particular in not well developed regions. The traditional detection typically involves measuring the mean corpuscular volume (i.e. the size of red blood cells) which may lead to the observation that the patient has a slightly decreased mean volume than normal. Furthermore, the patients typically have an increased fraction of hemoglobin A2 (>3.5%, for example 3.8% to 7%) and a decreased fraction of hemoglobin A (<97.5%). The genetic situation, or the mutations leading to thalassemia minor or beta thalassemia trait, are typically described as β⁺/β or β⁰/β. Due to the autosomal recessive inheritance of the disease beta thalassemia minor carriers, however, pose a major threat to public health since in subsequent generations combinations of recessive traits may lead to more severe forms of the disease.

In addition, further beta thalassemia variants are known, such as the Hb E/β⁰ thalassemia which is most prevalent in Thailand (Sherva et al., 2010, BMC Medical Genetics, 11, 51). In this variant a point mutation in codon 26 of the beta globin gene can induce alternative splicing which results in decreased beta globin E chains, leading to hypochromic microcytosis and minimal to severe anemia. Sherva et al. discovered 50 single nucleotide polymorphisms associated with this specific thalassemia form, which were mostly functionally linked to a regulatory region centromeric of the beta globin gene cluster.

The thalassemia forms are clustered in different geographical regions. Whereas alpha thalassemia is prevalent in West Africa and in the Americas, beta thalassemia can be found in populations in the Mediterranean region, in North Africa, West Asia and South Asia, which show the world's highest concentration of carriers. For example, in India, the carrier rate of beta thalassemia is assumed to be 3-17%.

It is assumed that there are 60-80 million people in the world who are beta thalassemia carriers. In particular, countries like India, Pakistan or Thailand are seeing a large increase of beta thalassemia patients due to a lack of genetic counseling and screening and there is growing concern that beta thalassemia may become a very serious problem in the next decades, which may, inter alia, burden the world's blood bank supplies and the health system in general.

Typically, the most valuable test for beta thalassemia carrier identification is the quantitative hemoglobin A2 determination, including, inter alia densitometry scanning after celluloase acetate electrophoresis, isoelectric focusing, capillary electrophoresis, hand high performance cation-exchange chromatography (HPLC). While the results of densitometry scanning are unsatisfactory, and isoelectric focusing is cumbersome and time-consuming, the superior HPLC-approach is mostly difficult to perform in regions without the necessary equipment.

There is, in consequence, a need for means and methods allowing for an easier, more straight forward, more sensitive and more specific screening and detection of beta thalassemia, in particular of beta thalassemia carriers.

### SUMMARY OF THE INVENTION

The present invention addresses this need and provides means and methods which allow the detection and identification of beta thalassemia, in particular of beta thalassemia carriers.

The above objective is in particular accomplished by an isolated nucleic acid molecule selected from the group comprising:

(i) SEQ ID NO: 1 [rs666247] except for a single polymorphic change at position 501, where wildtype nucleotide T is replaced by indicator nucleotide C;

(ii) SEQ ID NO: 2 [rs12707034] except for a single polymorphic change at position 501, where wildtype nucleotide T is replaced by indicator nucleotide C;

(iii) SEQ ID NO: 3 [rs707497] except for a single polymorphic change at position 501, where wildtype nucleotide T is replaced by indicator nucleotide C;

(iv) SEQ ID NO: 4 [rs17024172] except for a single polymorphic change at position 501, where wildtype nucleotide A is replaced by indicator nucleotide G;

(v) SEQ ID NO: 5 [rs16950705] except for a single polymorphic change at position 501, where wildtype nucleotide C is replaced by indicator nucleotide T;

(vi) SEQ ID NO: 6 [rs11956461] except for a single polymorphic change at position 501, where wildtype nucleotide C is replaced by indicator nucleotide T;

(vii) SEQ ID NO: 7 [rs609539] except for a single polymorphic change at position 501, where wildtype nucleotide G is replaced by indicator nucleotide A;

(viii) SEQ ID NO: 8 [rs7975838] except for a single polymorphic change at position 501, where wildtype nucleotide T is replaced by indicator nucleotide C;

(ix) SEQ ID NO: 9 [rs12063296] except for a single polymorphic change at position 501, where wildtype nucleotide A is replaced by indicator nucleotide G;

(x) SEQ ID NO: 10 [rs16913719] except for a single polymorphic change at position 501, where wildtype nucleotide C is replaced by indicator nucleotide T;

(xi) SEQ ID NO: 11 [rs11497898] except for a single polymorphic change at position 501, where wildtype nucleotide T is replaced by indicator nucleotide C;

(xii) SEQ ID NO: 12 [rs17168572] except for a single polymorphic change at position 501, where wildtype nucleotide A is replaced by indicator nucleotide G;

(xiii) SEQ ID NO: 13 [rs16933412] except for a single polymorphic change at position 501, where wildtype nucleotide T is replaced by indicator nucleotide C; and

(xiv) SEQ ID NO: 14 [rs16864505] except for a single polymorphic change at position 501, where wildtype nucleotide C is replaced by indicator nucleotide T.

These sequences constitute novel single nucleotide polymorphisms (SNPs) associated with beta thalassemia. They accordingly allow a sensitive, specific, effective, and simple detection and diagnosis approach towards beta thalassemia, e.g. by the employment of wide spread and easy to use techniques such as PCR or nucleic acid hybridization, which are assumed to have a high applicability and availability rate, in particular in less developed regions of the world. The novel SNPs, which were identified in a genome wide association study (GWAS) with samples from a North Indian population based on high-throughput genotyping technologies offer the additional advantage of providing a better understanding of beta thalassemia pathophysiology, which may lead to an improved disease management, in particular with regard to population genetics aspects. In particular by largely being based on the phenotype of beta thalassemia minor, which was corroborated by HPLC analysis, the SNPs are very useful for the detection of this beta thalassemia variant, i.e. for the identification of beta thalassemia carriers, which may otherwise be phenotypically rather unapparent. A corresponding genetics screening or counseling approach may be very helpful in confining the consequences of beta thalassemia as autosomal recessive disease. Furthermore, the SNPs allow the use of highly modern detection methods such as microarray analysis and genome sequencing, which may be implemented on a high-throughput basis. Finally, due to the fact that the SNPs were identified in an Indian population they allow the design of assays tailor-made for South Asian populations, in particular for the Indian population. The novel SNPs are thus considered to be useful for a population specific beta thalassemia detection for South Asian populations, in particular for the Indian population.

In a preferred embodiment of the present invention a panel of at least 1, 2, 3, 4, 5, 6, 7, 8, 9, 10, 11, 12, 13, or all of the above mentioned polymorphic, changed sequences comprising the above mentioned indicator nucleotides constitutes a marker for beta thalassemia. In a further preferred embodiment of the present invention a panel of at least 1, 2, 3, 4, 5, 6, 7, 8, 9, 10, 11, 12, 13, or all of the above mentioned polymorphic, changed sequences comprising the above mentioned indicator nucleotides constitutes a marker for beta thalassemia minor.

In yet another preferred embodiment the present invention relates the isolated nucleic acid or group or panel of nucleic acids as mentioned above, wherein said panel comprises at least:

(i) SEQ ID NO: 1 except for a single polymorphic change at position 501, where wildtype nucleotide T is replaced by indicator nucleotide C; or

(ii) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; or

(iii) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; or

(iv) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; or

(v) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 5 except for a single polymorphic change at position 501, where wildtype nucleotide C is replaced by indicator nucleotide T; or

(vi) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 5 except for a single polymorphic change at position 501, where wildtype nucleotide C is replaced by indicator nucleotide T; and SEQ ID NO: 6 except for a single polymorphic change at position 501, where wildtype nucleotide C is replaced by indicator nucleotide T; or

(vii) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 5 except for a single polymorphic change at position 501, where wildtype nucleotide C is replaced by indicator nucleotide T; and SEQ ID NO: 6 except for a single polymorphic change at position 501, where wildtype nucleotide C is replaced by indicator nucleotide T; and SEQ ID NO: 7 except for a single polymorphic change at position 501, where wildtype nucleotide G is replaced by indicator nucleotide A; or

(viii) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 5 except for a single polymorphic change at position 501, where wildtype nucleotide C is replaced by indicator nucleotide T; and SEQ ID NO: 6 except for a single polymorphic change at position 501, where wildtype nucleotide C is replaced by indicator nucleotide T; and SEQ ID NO: 7 except for a single polymorphic change at position 501, where wildtype nucleotide G is replaced by indicator nucleotide A; and SEQ ID NO: 8 except for a single polymorphic change at position 501, where wildtype nucleotide T is replaced by indicator nucleotide C; and SEQ ID NO: 9 except for a single polymorphic change at position 501, where wildtype nucleotide A is replaced by indicator nucleotide G; and SEQ ID NO: 10 except for a single polymorphic change at position 501, where wildtype nucleotide C is replaced by indicator nucleotide T; and SEQ ID NO: 11 except for a single polymorphic change at position 501, where wildtype nucleotide T is replaced by indicator nucleotide C; and SEQ ID NO: 12 except for a single polymorphic change at position 501, where wildtype nucleotide A is replaced by indicator nucleotide G; and SEQ ID NO: 13 except for a single polymorphic change at position 501, where wildtype nucleotide T is replaced by indicator nucleotide C; and SEQ ID NO: 14 except for a single polymorphic change at position 501, where wildtype nucleotide C is replaced by indicator nucleotide T; or

(ix) SEQ ID NO: 8 except for a single polymorphic change at position 501, where wildtype nucleotide T is replaced by indicator nucleotide C; and SEQ ID NO: 14 except for a single polymorphic change at position 501, where wildtype nucleotide C is replaced by indicator nucleotide T; or

(x) SEQ ID NO: 8 except for a single polymorphic change at position 501, where wildtype nucleotide T is replaced by indicator nucleotide C; and SEQ ID NO: 9 except for a single polymorphic change at position 501, where wildtype nucleotide A is replaced by indicator nucleotide G; or

(xi) SEQ ID NO: 2 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 13 except for a single polymorphic change at position 501, where wildtype nucleotide T is replaced by indicator nucleotide C.

In a further aspect the present invention relates to a method for detecting or diagnosing beta thalassemia in a subject comprising the steps of:

(a) isolating a nucleic acid from a subject's sample

(b) determining the nucleotide sequence and/or molecular structure present at one or more polymorphic sites as defined herein above;

wherein the presence of an indicator nucleotide as defined herein above is indicative of the presence of beta thalassemia.

In a preferred embodiment the present invention relates to method for detecting or diagnosing beta thalassemia minor in a subject comprising the steps of:

(a) isolating a nucleic acid from a subject's sample

(b) determining the nucleotide sequence and/or molecular structure present at one or more polymorphic sites as defined herein above;

wherein the presence of an indicator nucleotide as defined herein above is indicative of the presence of beta thalassemia minor.

In further preferred embodiment said determination of the nucleotide sequence may be carried out through allele-specific oligonucleotide (ASO)-dot blot analysis, primer extension assays, iPLEX SNP genotyping, Dynamic allele-specific hybridization (DASH) genotyping, the use of molecular beacons, tetra primer ARMS PCR, a flap endonuclease invader assay, an oligonucleotide ligase assay, PCR-single strand conformation polymorphism (SSCP) analysis, quantitative real-time PCR assay, SNP microarray based analysis, restriction enzyme fragment length polymorphism (RFLP) analysis, targeted resequencing analysis and/or whole genome sequencing analysis.

In yet another preferred embodiment of the present invention said method comprises as additional step the determination of the Hb A2 concentration in the sample.

In a further preferred embodiment said determination of Hb A2 concentration may be carried out via HPLC, microchromatography, isoelectric focusing, or capillary electrophoresis.

In another preferred embodiment of the present invention the above mentioned sample may be a mixture of tissues, organs, cells and/or fragments thereof, or a tissue or organ specific sample, such as a tissue biopsy from vaginal tissue, tongue, pancreas, liver, spleen, ovary, muscle, joint tissue, neural tissue, gastrointestinal tissue, tumor tissue, or a body fluid, blood, serum, saliva, or urine. Particularly preferred is blood.

In yet another preferred embodiment of the present invention the method as mentioned herein above comprises the determination of the nucleotide sequence and/or molecular structure present at polymorphic sites of SEQ ID NO: 8 and SEQ ID NO: 9 and the detection of a DNAse hypersensitivity site in the genomic vicinity of SEQ ID NO: 8 and/or SEQ ID NO: 9, wherein the presence of an indicator nucleotide as defined herein above and the presence of said DNAse hypersensitivity site is indicative of the presence of beta thalassemia.

In yet another preferred embodiment of the present invention the method as mentioned herein above comprises the determination of the nucleotide sequence and/or molecular structure present at polymorphic sites of SEQ ID NO: 2, SEQ ID NO: 4 and SEQ ID NO: 13 and the detection of a histone 3 lysine 27 trimethylation in the genomic vicinity of SEQ ID NO: 2 and/or SEQ ID NO: 4 and/or SEQ ID NO: 13, wherein the presence of an indicator nucleotide as defined herein above and the presence of said histone 3 lysine 27 trimethylation is indicative of the presence of beta thalassemia.

In another aspect the present invention relates to a composition for detecting or diagnosing beta thalassemia in a subject comprising a nucleic acid affinity ligand for one or more polymorphic sites as defined herein above.

In a preferred embodiment the present invention relates to a composition for detecting or diagnosing beta thalassemia minor in a subject comprising a nucleic acid affinity ligand for one or more polymorphic sites as defined herein above.

In yet another preferred embodiment of the present invention the affinity ligand as mentioned herein above may be an oligonucleotide specific for one or more polymorphic sites as defined herein above, or a probe specific for one or more polymorphic sites as defined herein above. In a particularly preferred embodiment of the present invention the affinity ligand as mentioned herein above may be an oligonucleotide having a sequence complementary to an indicator nucleotide as defined herein above.

In another aspect the present invention relates to the use of a nucleic acid molecule as defined herein above for detecting or diagnosing beta thalassemia in a subject, or for screening a population of subjects for the presence of beta thalassemia. In a particularly preferred embodiment said beta thalassemia may be beta thalassemia minor. In a further particularly preferred embodiment said population of subjects may be a South Asian population of subjects.

In another aspect the present invention relates to a kit for detecting or diagnosing beta thalassemia in a subject, comprising an oligonucleotide specific for one or more polymorphic sites as defined herein above, or a probe specific for one or more polymorphic sites as defined herein above. In a particularly preferred embodiment said oligonucleotide has a sequence complementary to an indicator nucleotide as defined herein above. In another particularly preferred embodiment said beta thalassemia is beta thalassemia minor.

In yet another preferred embodiment the above mentioned method, composition, use, or kit relate to the assessment of the risk of developing beta thalassemia in a subject and/or in a subject's progeny.

### BRIEF DESCRIPTION OF THE DRAWINGS

FIG. 1 shows hemoglobin A2 indicated in % for case and control subjects as detected by HPLC.

FIG. 2 provides an overall scheme of the genome wide association study for beta thalassemia minor.

FIG. 3 shows a workflow for genotyping and downstream analysis based on the use of Affymetrix SNP 6.0.

FIG. 4 shows the results of a quality control analysis for all samples obtained in the genotyping console. The threshold was set as 86%. All 48 cases and 66 controls had QC call rate above 86%.

FIG. 5 shows a plot of p-values of the identified SNPs against the chromosomes of the human cell.

FIG. 6 shows haplotype blocks capturing associated SNPs.

FIG. 6 shows the haplotype blocks of chromosome 1,

FIG. 6B shows the haplotype blocks of chromosome 2,

FIG. 6C shows the haplotype blocks of chromosome 5,

FIG. 6D shows the haplotype blocks of chromosome 6,

FIG. 6E shows the haplotype blocks of chromosome 7,

FIG. 6F shows the haplotype blocks of chromosome 8;

FIG. 6G shows the haplotype blocks of chromosome 10 and

FIG. 6H shows the haplotype blocks of chromosome 12.

### DETAILED DESCRIPTION OF EMBODIMENTS

The inventors have developed means and methods which allow the detection and identification of beta thalassemia, in particular of beta thalassemia minor and beta thalassemia carriers.

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In case the terms “first”, “second”, “third” or “(a)”, “(b)”, “(c)”, “(d)” etc. relate to steps of a method or use there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As has been set out above, the present invention concerns in one aspect an isolated nucleic acid molecule selected from the group comprising:

(i) SEQ ID NO: 1 [rs666247] except for a single polymorphic change at position 501, where wildtype nucleotide T is replaced by indicator nucleotide C;

(ii) SEQ ID NO: 2 [rs12707034] except for a single polymorphic change at position 501, where wildtype nucleotide T is replaced by indicator nucleotide C;

(iii) SEQ ID NO: 3 [rs707497] except for a single polymorphic change at position 501, where wildtype nucleotide T is replaced by indicator nucleotide C;

(iv) SEQ ID NO: 4 [rs17024172] except for a single polymorphic change at position 501, where wildtype nucleotide A is replaced by indicator nucleotide G;

(v) SEQ ID NO: 5 [rs16950705] except for a single polymorphic change at position 501, where wildtype nucleotide C is replaced by indicator nucleotide T;

(vi) SEQ ID NO: 6 [rs11956461] except for a single polymorphic change at position 501, where wildtype nucleotide C is replaced by indicator nucleotide T;

(vii) SEQ ID NO: 7 [rs609539] except for a single polymorphic change at position 501, where wildtype nucleotide G is replaced by indicator nucleotide A;

(viii) SEQ ID NO: 8 [rs7975838] except for a single polymorphic change at position 501, where wildtype nucleotide T is replaced by indicator nucleotide C;

(ix) SEQ ID NO: 9 [rs12063296] except for a single polymorphic change at position 501, where wildtype nucleotide A is replaced by indicator nucleotide G;

(x) SEQ ID NO: 10 [rs16913719] except for a single polymorphic change at position 501, where wildtype nucleotide C is replaced by indicator nucleotide T;

(xi) SEQ ID NO: 11 [rs11497898] except for a single polymorphic change at position 501, where wildtype nucleotide T is replaced by indicator nucleotide C;

(xii) SEQ ID NO: 12 [rs17168572] except for a single polymorphic change at position 501, where wildtype nucleotide A is replaced by indicator nucleotide G;

(xiii) SEQ ID NO: 13 [rs16933412] except for a single polymorphic change at position 501, where wildtype nucleotide T is replaced by indicator nucleotide C; and

(xiv) SEQ ID NO: 14 [rs16864505] except for a single polymorphic change at position 501, where wildtype nucleotide C is replaced by indicator nucleotide T.

The term “isolated nucleic acid molecule” a used herein refers to a nucleic acid entity, e.g. DNA, RNA etc, wherein the entity is substantially free of other biological molecules, such as nucleic acids, proteins, lipids, carbohydrates, or other material, such as cellular debris and growth media. Generally the term “isolated” is not intended to refer to the complete absence of such material, or to the absence of water, buffers, or salts, unless they are present in amounts which substantially interfere with the methods of the present invention.

The term “wildtype sequence” as mentioned herein refers to the sequence of an allele, which does not show the associated phenotype according to the present invention, preferably which does not show the associated phenotype of beta thalassemia. The term may further refer to the sequence of the non phenotype-associated allele with the highest prevalence within a population, preferably within a South Asian population, more preferably within the Indian population.

The term “indicator sequence” as used herein refers to the sequence of an allele, which shows an association with a phenotype according to the present invention. Preferably, it shows an association with the phenotype of beta thalassemia. In specific embodiments of the present invention, an indicator sequence may be not only the above indicated allelic sequence for each of SEQ ID NO: 1 to 14, but also an independent, further variation from the wildtype sequence as defined herein.

The term “allele” or “allelic sequence” as used herein refers to a particular form of a gene or a particular nucleotide, preferably a DNA sequence at a specific chromosomal location or locus. In certain embodiments of the present invention a SNP as defined herein may be found at one of two alleles in the human genome of a single subject. In further, specific embodiments, a SNP as defined herein may also be found at both alleles in the human genome of a single subject.

SEQ ID NOs: 1 to 14 as mentioned above comprise a stretch of 1001 nucleotides each and represent the wildtype sequence at an encountered polymorphic site with 500 nucleotides context sequence upstream and downstream thereof. The present invention accordingly envisages the sequence depicted in SEQ ID NO: 1 to 14, in particular the polymorphic nucleotides at position 501 thereof, as well as the sequence of the complementary strand of SEQ ID NO: 1 to 14, in particular the polymorphic nucleotides at position 501 of said complementary strand. For analytic purposes the strand identity may be define, or fixed, or may be choose at will, e.g. in dependence on factors such the availability of binding elements, GC-content etc. Furthermore, for the sake of accuracy, the SNP may be defined on both strands at the same time, and accordingly be analyzed.

SEQ ID NO: 1 as mentioned herein defines a sequence of single nucleotide polymorphism (SNP) rs666247, which is located on chromosome 6, cytoband p22.3, position 20032959-20033959 according to NCBI build 37.1 of the human genome, wherein at position 501 the wildtype nucleotide T is replaced by an indicator nucleotide, preferably by the nucleotide C. The SNP shows a minor allele frequency of 0.21559633. The SNP locus is located in the vicinity of gene LOC729105 at a distance of 23967 and 13778. The distance indicates the maximum distance on both sides of the SNP.

SEQ ID NO: 2 as mentioned herein defines a sequence of single nucleotide polymorphism (SNP) rs12707034, which is located on chromosome 7, cytoband q32.3, position 132016658-132017658 according to NCBI build 37.1 of the human genome, wherein at position 501 the wildtype nucleotide T is replaced by an indicator nucleotide, preferably by the nucleotide C. The SNP shows a minor allele frequency of 0.233009709. The SNP locus is located in the vicinity of gene PLXNA4 at a distance of 209067 and 316289.

SEQ ID NO: 3 as mentioned herein defines a sequence of single nucleotide polymorphism (SNP) rs707497, which is located on chromosome 2, cytoband q14.3, position 125064809-125065809 according to NCBI build 37.1 of the human genome, wherein at position 501 the wildtype nucleotide T is replaced by an indicator nucleotide, preferably by the nucleotide C. The SNP shows a minor allele frequency of 0.223300971. The SNP locus is located in the vicinity of gene CNTNAP5 at a distance of 282445 and 607555.

SEQ ID NO: 4 as mentioned herein defines a sequence of single nucleotide polymorphism (SNP) rs17024172, which is located on chromosome 2, cytoband p22.1, position 39931763-39932763 according to NCBI build 37.1 of the human genome, wherein at position 501 the wildtype nucleotide A is replaced by an indicator nucleotide, preferably by the nucleotide G. The SNP shows a minor allele frequency of 0.186363636. The SNP locus is located in the vicinity of gene TMEM178 at a distance of 39173 and 1284.

SEQ ID NO: 5 as mentioned herein defines a sequence of single nucleotide polymorphism (SNP) rs16950705, which is located on chromosome 16, cytoband q12.1, position 52061759-52062759 according to NCBI build 37.1 of the human genome, wherein at position 501 the wildtype nucleotide C is replaced by an indicator nucleotide, preferably by the nucleotide T. The SNP shows a minor allele frequency of 0.183962264. The SNP locus is located in the vicinity of gene LOC388276 at a distance of 1995 and 46604.

SEQ ID NO: 6 as mentioned herein defines a sequence of single nucleotide polymorphism (SNP) rs11956461, which is located on chromosome 5, cytoband q21.2, position 104378123-104379123 according to NCBI build 37.1 of the human genome, wherein at position 501 the wildtype nucleotide C is replaced by an indicator nucleotide, preferably by the nucleotide T. The SNP shows a minor allele frequency of 0.160377358. The SNP locus is located in the vicinity of genes NUDT12 and RAB9P1 at distances of −1480133 and −56552, respectively.

SEQ ID NO: 7 as mentioned herein defines a sequence of single nucleotide polymorphism (SNP) rs609539, which is located on chromosome 5, cytoband q21.3, position 106904497-106905497 according to NCBI build 37.1 of the human genome, wherein at position 501 the wildtype nucleotide G is replaced by an indicator nucleotide, preferably by the nucleotide A. The SNP shows a minor allele frequency of 0.291262136. The SNP locus is located in the vicinity of gene EFNA5 at a distance of 188646 and 101599.

SEQ ID NO: 8 as mentioned herein defines a sequence of single nucleotide polymorphism (SNP) rs7975838, which is located on chromosome 12, cytoband q24.22, position 116881224-116882224 according to NCBI build 37.1 of the human genome, wherein at position 501 the wildtype nucleotide T is replaced by an indicator nucleotide, preferably by the nucleotide C. The SNP shows a minor allele frequency of 0.327102804. The SNP locus is located in the vicinity of genes MED13L and F1142957 at distances of −166581 and −89503, respectively.

SEQ ID NO: 9 as mentioned herein defines a sequence of single nucleotide polymorphism (SNP) rs12063296, which is located on chromosome 1, cytoband q25.1, position 173929399-173930399 according to NCBI build 37.1 of the human genome, wherein at position 501 the wildtype nucleotide A is replaced by an indicator nucleotide, preferably by the nucleotide G. The SNP shows a minor allele frequency of 0.132075472. The SNP locus is located in the vicinity of gene RC3H1 at a distance of 29547 and 32311.

SEQ ID NO: 10 as mentioned herein defines a sequence of single nucleotide polymorphism (SNP) rs16913719, which is located on chromosome 9, cytoband p21.1, position 28819174-28820174 according to NCBI build 37.1 of the human genome, wherein at position 501 the wildtype nucleotide C is replaced by an indicator nucleotide, preferably by the nucleotide T. The SNP shows a minor allele frequency of 0.14159292. The SNP locus is located in the vicinity of genes LOC646700 and MIRN876, at distances of −670440 and −43949, respectively.

SEQ ID NO: 11 as mentioned herein defines a sequence of single nucleotide polymorphism (SNP) rs11497898, which is located on chromosome 10, cytoband q21.3, position 66518352-66519352 according to NCBI build 37.1 of the human genome, wherein at position 501 the wildtype nucleotide T is replaced by an indicator nucleotide, preferably by the nucleotide C. The SNP shows a minor allele frequency of 0.135514019. The SNP locus is located in the vicinity of genes LOC100129267 and ANXA2P3, at distances of −587977 and −66433, respectively.

SEQ ID NO: 12 as mentioned herein defines a sequence of single nucleotide polymorphism (SNP) rs17168572, which is located on chromosome 7, cytoband q21.3, position 97065519-97066519 according to NCBI build 37.1 of the human genome, wherein at position 501 the wildtype nucleotide A is replaced by an indicator nucleotide, preferably by the nucleotide G. The SNP shows a minor allele frequency of 0.133027523. The SNP locus is located in the vicinity of genes LOC442712 and TAC1, at distances of −235259 and −295356, respectively.

SEQ ID NO: 13 as mentioned herein defines a sequence of single nucleotide polymorphism (SNP) rs16933412, which is located on chromosome 8, cytoband q13.2, position 68497405-68498405 according to NCBI build 37.1 of the human genome, wherein at position 501 the wildtype nucleotide T is replaced by an indicator nucleotide, preferably by the nucleotide C. The SNP shows a minor allele frequency of 0.169724771. The SNP locus is located in the vicinity of gene CPA6, at a distance of 163497 and 160675.

SEQ ID NO: 14 as mentioned herein defines a sequence of single nucleotide polymorphism (SNP) rs16864505, which is located on chromosome 2, cytoband q36.1, position 224018270-224019270 according to NCBI build 37.1 of the human genome, wherein at position 501 the wildtype nucleotide C is replaced by an indicator nucleotide, preferably by the nucleotide T. The SNP shows a minor allele frequency of 0.199029126. The SNP locus is located in the vicinity of genes KCNE4 and SCG2, at distances of −98415 and −442888, respectively.

In specific embodiments of the present invention the envisaged nucleic acid molecules comprise sequences of SEQ ID NO: 1 to 14, essentially consist of sequences of SEQ ID NO: 1 to 14, or consist of sequences of SEQ ID NO: 1 to 14. For example, the sequences may comprise adjacent regions in the 3′ and/or 5′ context of SEQ ID NO: 1 to 14, as defined herein, e.g. stretch for additional about 100, 200, 300, 400, 500, 600, 700, 800, 900, 1000, 1500, 2000, 3000, 4000, 5000, 6000, 7000, 10 000 or more nucleotides into 3′ and/or 5′ direction starting from the herein above indicated genomic positions.

In further embodiments of the present invention the envisaged nucleic acid molecules may comprise, essentially consist of, or consist of fragments of SEQ ID NO: 1 to 14 which at least have to comprise the polymorphic sites at position 501 of SEQ ID NO: 1 to 14. For example, the present invention relates to sequences of about 900, 800, 700, 600, 500, 400, 300, 200, 100, 90, 80, 70, 60, 50, 40, 30, 20, or 10 or less nucleotides length or any value in between, which have to comprise at least the polymorphic sites at position 501 of SEQ ID NO: 1 to 14. The fragments may extend for the indicated length towards the 5′ or 3′ direction, or in both, the 5′ and 3′ direction. Preferred are fragments of a length of 100 nucleotides and less with the SNPs at around position 50 or a corresponding position, i.e. in the center of the sequence.

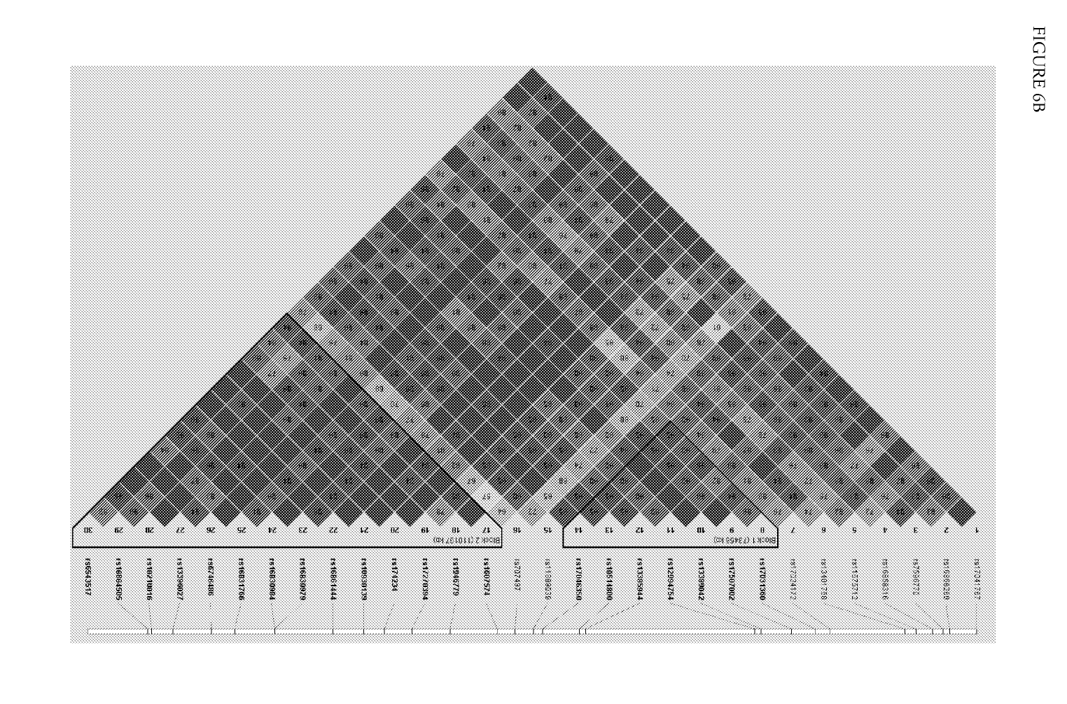

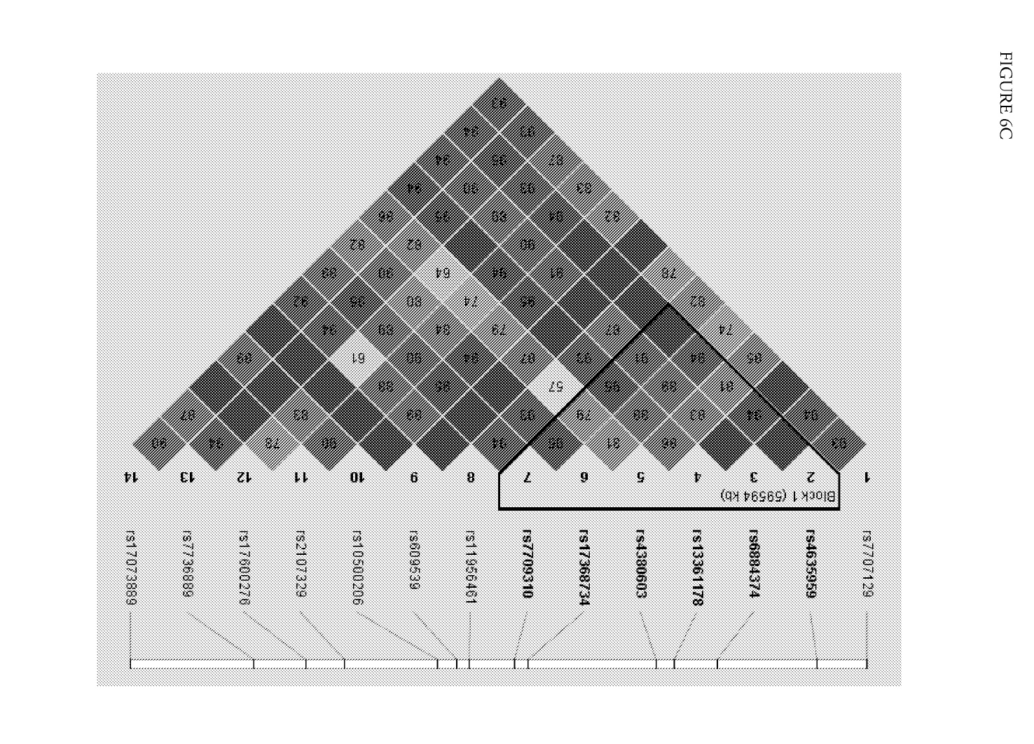

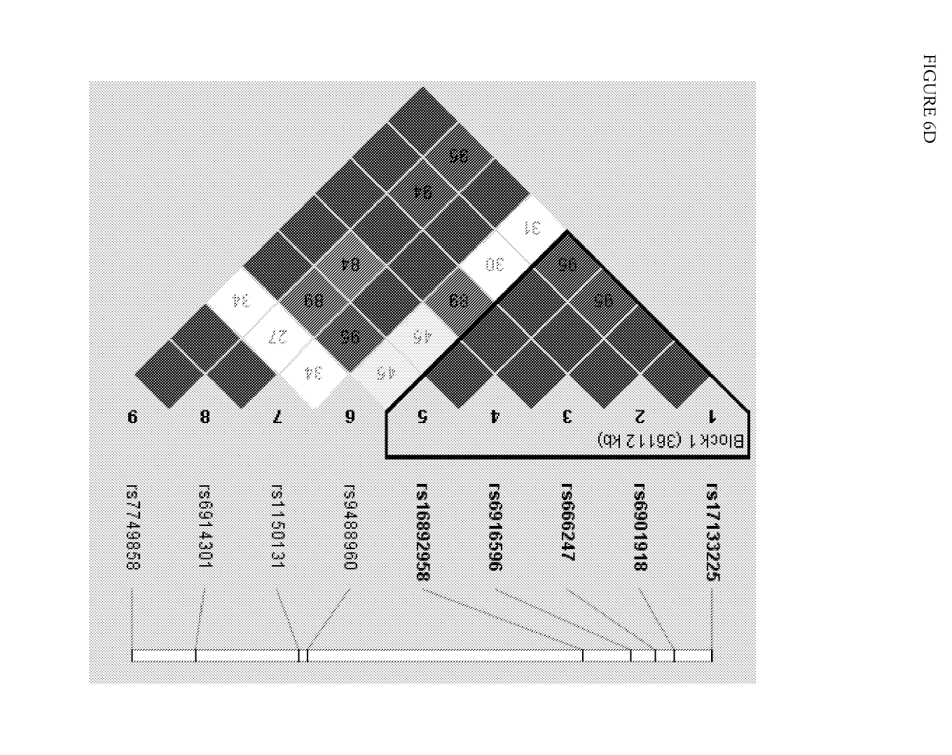

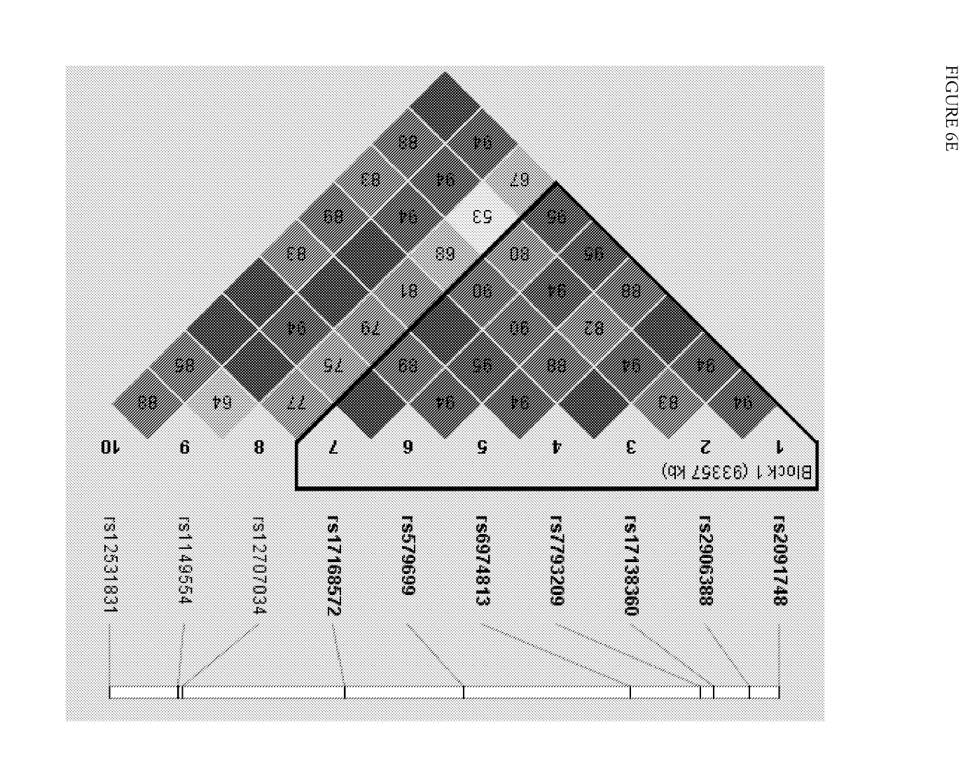

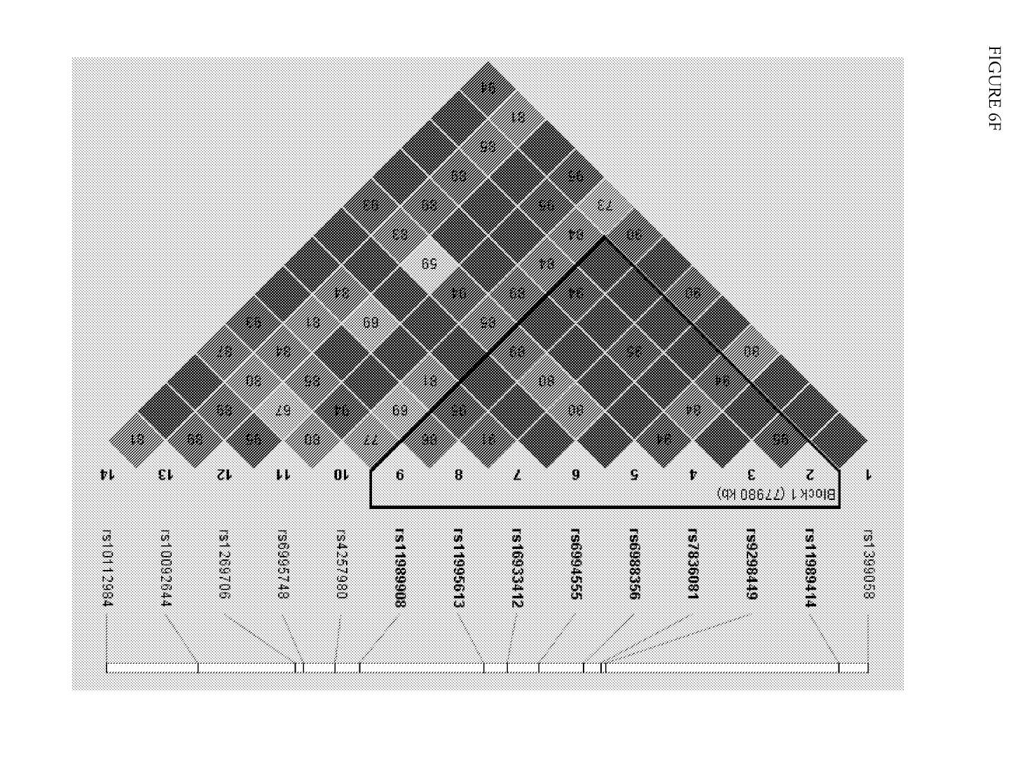

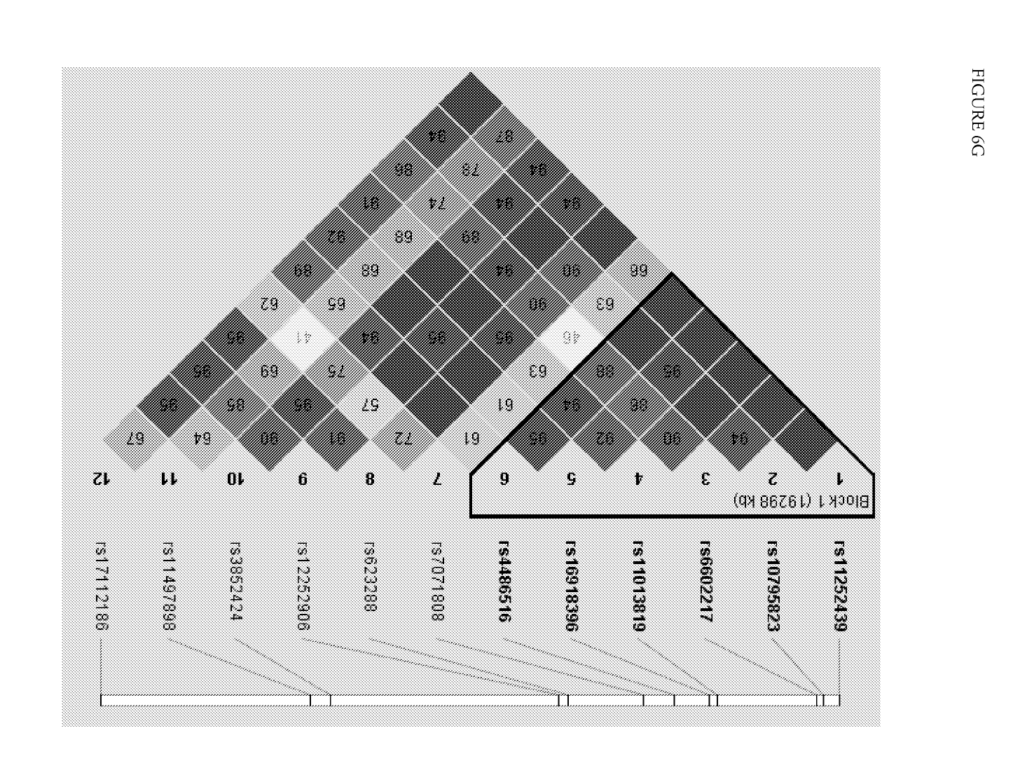

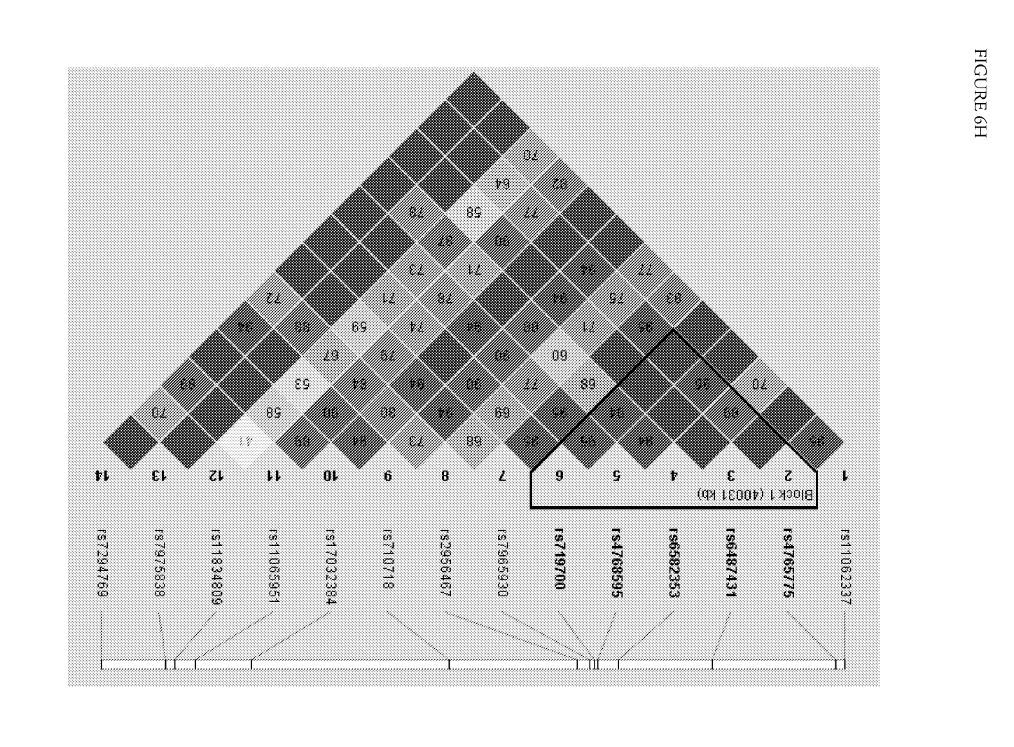

In further embodiments the present invention also encompasses haplotypes including one or more of the SNPs as defined herein above, i.e. SEQ ID NO: 1 with SNP rs666247, SEQ ID NO: 2 with SNP rs12707034, SEQ ID NO: 3 with SNP rs707497, SEQ ID NO: 4 with SNP rs17024172, SEQ ID NO: 5 with SNP rs16950705, SEQ ID NO: 6 with SNP rs11956461, SEQ ID NO: 7 with SNP rs609539, SEQ ID NO: 8 with SNP rs7975838, SEQ ID NO: 9 with SNP rs12063296, SEQ ID NO: 10 with SNP rs16913719, SEQ ID NO: 11 with SNP rs11497898, SEQ ID NO: 12 with SNP rs17168572, SEQ ID NO: 13 with SNP rs16933412, or SEQ ID NO: 14 with SNP rs16864505. The term “haplotype” as used herein refers to a 5′ to 3′ sequence of nucleotides found at one or more linked polymorphic sites in a locus on a single chromosome from a single subject. Preferably, the present invention encompasses haplotypes as defined in Example 2 or as shown in FIG. 6 A to H, or haplotypes as mentioned in Table 6. Particularly preferred are haplotypes showing a p-value of ≦10⁻¹⁰, as derivable from Table 6.

More preferably, the present invention relates to the following haplotypes:

(i) for chromosome 1: rs11573269 (SEQ ID NO: 9), rs4654885, rs441380, rs10493137, rs6657279, rs6683003, rs12082126, rs1529594, rs12087676, rs11209819, rs576056, rs11808445, rs698944, rs291565, rs17120268, rs41343145, rs17018484, rs16857061, rs12131192, rs12063296, rs10913087, rs3009323, rs805911, rs6701222, rs1389970, rs6693224, rs6667309;

(ii) for chromosome 2: rs1607574, rs1946779, rs17270394, rs174234, rs10930139, rs16861444, rs16830979, rs16830984, rs16831766, rs6746486, rs13396027, rs10210016, rs16864505 (SEQ ID NO: 14), rs6543517;

(iii) for chromosome 6: rs17133225, rs6901918, rs666247 (SEQ ID NO: 1), rs6916596, rs16892958;

(iv) for chromosome 7: rs2091148, rs2906388, rs17138360, rs7793209, rs6974813, rs579699, rs17168572 (SEQ ID NO: 12); and

(v) for chromosome 8: rs11989414, rs9298449, rs7836081, rs6988356, rs6994555, rs16933412 (SEQ ID NO: 13), rs11995613, rs11989908.

A person skilled in the art would accordingly be able to derive the exact position, nucleotide sequence, and indicator sequence from the above identified rs-nomenclature, e.g. from suitable database entries and associated information systems, e.g. the Single Nucleotide Polymorphism database (dbSNP) which is incorporated herein by reference.

In further embodiment the present invention relates to one or more, e.g. a panel, of the above mentioned polymorphic, changed sequences comprising the above mentioned indicator nucleotides, as constituting a marker for beta thalassemia. The term “marker for beta thalassemia” as used herein refers to the association of the mentioned SNP comprising the above identified indicator nucleotide at a sequence position as defined herein above in at least one allele, or, in specific embodiment, in two alleles of single subject, and the disease beta thalassemia. Thus, a subject comprising or showing one or more of the SNPs as defined herein above, in particular the SNPs as defined in the context of SEQ ID NO: 1 to 14 with the correspondingly identified indicator nucleotides may be considered as being affected by beta thalassemia. The term “beta thalassemia” as used herein refers to one or more genetic modifications, typically an autosomal recessive mutation, which leads to the absence or reduction of amount of the beta hemoglobin protein in a subject. The disease may be present in the form of thalassemia intermedia or, preferably, thalassemia minor, or in exceptional cases thalassemia major, showing, for example, one of the possible genetic situations β⁺/β, β⁰/β, β⁺/β⁰, β⁰/β⁰, β⁺/β⁺, wherein “β” describes alleles without a mutation that reduces the function of beta hemoglobin, “β⁺” describes alleles comprising mutations which allow some beta hemoglobin chains formation to occur and “β⁰” describes alleles comprising mutations which entirely prevent the production of beta hemoglobin chains.

In a further preferred embodiment the present invention relates to one or more, e.g. a panel, of the above mentioned polymorphic, changed sequences comprising the above mentioned indicator nucleotides, as constituting a marker for beta thalassemia minor. The term “beta thalassemia minor” as used herein refers to possible genetic situations of β⁺/β or β⁰/β. This disorder is characterized by a mild to moderate anemia, which is typically not life threatening. The disorder may further show the phenotype of an increased fraction of hemoglobin A2 (>3.5%, for example 3.8% to 7%) and a decreased fraction of hemoglobin A (<97.5%). Since subjects afflicted by beta thalassemia minor are carriers of the autosomal recessive trait of beta thalassemia, the one or more, e.g. a panel, of the above mentioned polymorphic, changed sequences comprising the above mentioned indicator nucleotides also preferably constitute markers or identifiers for beta thalassemia carriers. The term “beta thalassemia” as used herein accordingly also includes the beta thalassemia carrier situation as mentioned above which may be apparent or, in other embodiments, possibly be unapparent.

In further preferred embodiments 1, 2, 3, 4, 5, 6, 7, 8, 9, 10, 11, 12, 13, or all of the above mentioned polymorphic, changed sequences comprising the above mentioned indicator nucleotides may constitute the marker. Preferably, a single SNPs may be used as a marker for beta thalassemia as defined herein above, preferably for beta thalassemia minor or for beta thalassemia carriers as mentioned herein above. Also preferred are combinations of any possible 2 of the SNPs of the present invention, e.g. SEQ ID NO: 1 with SNP rs666247 and SEQ ID NO: 2 with SNP rs12707034, SEQ ID NO: 3 with SNP rs707497 and SEQ ID NO: 4 with SNP rs17024172, SEQ ID NO: 5 with SNP rs16950705 and SEQ ID NO: 6 with SNP rs11956461, SEQ ID NO: 7 with SNP rs609539 and SEQ ID NO: 8 with SNP rs7975838, SEQ ID NO: 9 with SNP rs12063296 and SEQ ID NO: 10 with SNP rs16913719, SEQ ID NO: 11 with SNP rs11497898 and SEQ ID NO: 12 with SNP rs17168572, or SEQ ID NO: 13 with SNP rs16933412 and SEQ ID NO: 14 with SNP rs16864505 etc. Further envisaged are all other 2 SNP permutations or groupings of the mentioned SNPs.

Further preferred are combinations or panels of any possible 3 of the SNPs of the present invention, e.g. SEQ ID NO: 1 with SNP rs666247 and SEQ ID NO: 2 with SNP rs12707034 and SEQ ID NO: 3 with SNP rs707497, SEQ ID NO: 4 with SNP rs17024172 and SEQ ID NO: 5 with SNP rs16950705 and SEQ ID NO: 6 with SNP rs11956461, SEQ ID NO: 7 with SNP rs609539 and SEQ ID NO: 8 with SNP rs7975838 and SEQ ID NO: 9 with SNP rs12063296, SEQ ID NO: 10 with SNP rs16913719 and SEQ ID NO: 11 with SNP rs11497898 and SEQ ID NO: 12 with SNP rs17168572, or SEQ ID NO: 13 with SNP rs16933412 and SEQ ID NO: 14 with SNP rs16864505 and SEQ ID NO: 1 with SNP rs666247 etc. Further envisaged are all other 3 SNP permutations or groupings of the mentioned SNPs.

Further preferred are combinations or panels of any possible 4 of the SNPs of the present invention, e.g. SEQ ID NO: 1 with SNP rs666247 and SEQ ID NO: 2 with SNP rs12707034 and SEQ ID NO: 3 with SNP rs707497 and SEQ ID NO: 4 with SNP rs17024172; SEQ ID NO: 5 with SNP rs16950705 and SEQ ID NO: 6 with SNP rs11956461 and SEQ ID NO: 7 with SNP rs609539 and SEQ ID NO: 8 with SNP rs7975838; SEQ ID NO: 9 with SNP rs12063296 and SEQ ID NO: 10 with SNP rs16913719 and SEQ ID NO: 11 with SNP rs11497898 and SEQ ID NO: 12 with SNP rs17168572, or SEQ ID NO: 13 with SNP rs16933412 and SEQ ID NO: 14 with SNP rs16864505 and SEQ ID NO: 1 with SNP rs666247 and SEQ ID NO: 2 with SNP rs12707034 etc. Further envisaged are all other 4 SNP permutations or groupings of the mentioned SNPs.

Further preferred are combinations or panels of any possible 5 of the SNPs of the present invention, e.g. SEQ ID NO: 1 with SNP rs666247 and SEQ ID NO: 2 with SNP rs12707034 and SEQ ID NO: 3 with SNP rs707497 and SEQ ID NO: 4 with SNP rs17024172 and SEQ ID NO: 5 with SNP rs16950705, SEQ ID NO: 6 with SNP rs11956461 and SEQ ID NO: 7 with SNP rs609539 and SEQ ID NO: 8 with SNP rs7975838 and SEQ ID NO: 9 with SNP rs12063296 and SEQ ID NO: 10 with SNP rs16913719; or SEQ ID NO: 11 with SNP rs11497898 and SEQ ID NO: 12 with SNP rs17168572 and SEQ ID NO: 13 with SNP rs16933412 and SEQ ID NO: 14 with SNP rs16864505 and SEQ ID NO: 1 with SNP rs666247 etc. Further envisaged are all other 5 SNP permutations or groupings of the mentioned SNPs.

Further preferred are combinations or panels of any possible 6 of the SNPs of the present invention, e.g. SEQ ID NO: 1 with SNP rs666247 and SEQ ID NO: 2 with SNP rs12707034 and SEQ ID NO: 3 with SNP rs707497 and SEQ ID NO: 4 with SNP rs17024172 and SEQ ID NO: 5 with SNP rs16950705 and SEQ ID NO: 6 with SNP rs11956461; SEQ ID NO: 7 with SNP rs609539 and SEQ ID NO: 8 with SNP rs7975838 and SEQ ID NO: 9 with SNP rs12063296 and SEQ ID NO: 10 with SNP rs16913719 and SEQ ID NO: 11 with SNP rs11497898 and SEQ ID NO: 12 with SNP rs17168572; or SEQ ID NO: 13 with SNP rs16933412 and SEQ ID NO: 14 with SNP rs16864505 and SEQ ID NO: 1 with SNP rs666247 and SEQ ID NO: 2 with SNP rs12707034 and SEQ ID NO: 3 with SNP rs707497 and SEQ ID NO: 4 with SNP rs17024172 etc. Further envisaged are all other 6 SNP permutations or groupings of the mentioned SNPs.

Further preferred are combinations or panels of any possible 7 of the SNPs of the present invention, e.g. SEQ ID NO: 1 with SNP rs666247 and SEQ ID NO: 2 with SNP rs12707034 and SEQ ID NO: 3 with SNP rs707497 and SEQ ID NO: 4 with SNP rs17024172 and SEQ ID NO: 5 with SNP rs16950705 and SEQ ID NO: 6 with SNP rs11956461 and SEQ ID NO: 7 with SNP rs609539; or SEQ ID NO: 8 with SNP rs7975838 and SEQ ID NO: 9 with SNP rs12063296 and SEQ ID NO: 10 with SNP rs16913719 and SEQ ID NO: 11 with SNP rs11497898 and SEQ ID NO: 12 with SNP rs17168572 and SEQ ID NO: 13 with SNP rs16933412 and SEQ ID NO: 14 with SNP rs16864505 etc. Further envisaged are all other 7 SNP permutations or groupings of the mentioned SNPs.

Further preferred are combinations or panels of any possible 8 of the SNPs of the present invention, e.g. SEQ ID NO: 1 with SNP rs666247 and SEQ ID NO: 2 with SNP rs12707034 and SEQ ID NO: 3 with SNP rs707497 and SEQ ID NO: 4 with SNP rs17024172 and SEQ ID NO: 5 with SNP rs16950705 and SEQ ID NO: 6 with SNP rs11956461 and SEQ ID NO: 7 with SNP rs609539 and SEQ ID NO: 8 with SNP rs7975838; or SEQ ID NO: 9 with SNP rs12063296 and SEQ ID NO: 10 with SNP rs16913719 and SEQ ID NO: 11 with SNP rs11497898 and SEQ ID NO: 12 with SNP rs17168572 and SEQ ID NO: 13 with SNP rs16933412 and SEQ ID NO: 14 with SNP rs16864505 and SEQ ID NO: 1 with SNP rs666247 and SEQ ID NO: 2 with SNP rs12707034 etc. Further envisaged are all other 8 SNP permutations or groupings of the mentioned SNPs.

Further preferred are combinations or panels of any possible 9 of the SNPs of the present invention, e.g. SEQ ID NO: 1 with SNP rs666247 and SEQ ID NO: 2 with SNP rs12707034 and SEQ ID NO: 3 with SNP rs707497 and SEQ ID NO: 4 with SNP rs17024172 and SEQ ID NO: 5 with SNP rs16950705 and SEQ ID NO: 6 with SNP rs11956461 and SEQ ID NO: 7 with SNP rs609539 and SEQ ID NO: 8 with SNP rs7975838 and SEQ ID NO: 9 with SNP rs12063296; or SEQ ID NO: 10 with SNP rs16913719 and SEQ ID NO: 11 with SNP rs11497898 and SEQ ID NO: 12 with SNP rs17168572 and SEQ ID NO: 13 with SNP rs16933412 and SEQ ID NO: 14 with SNP rs16864505 and SEQ ID NO: 1 with SNP rs666247 and SEQ ID NO: 2 with SNP rs12707034 and SEQ ID NO: 3 with SNP rs707497 and SEQ ID NO: 4 with SNP rs17024172 etc. Further envisaged are all other 9 SNP permutations or groupings of the mentioned SNPs.

Further preferred are combinations or panels of any possible 10 of the SNPs of the present invention, e.g. SEQ ID NO: 1 with SNP rs666247 and SEQ ID NO: 2 with SNP rs12707034 and SEQ ID NO: 3 with SNP rs707497 and SEQ ID NO: 4 with SNP rs17024172 and SEQ ID NO: 5 with SNP rs16950705 and SEQ ID NO: 6 with SNP rs11956461 and SEQ ID NO: 7 with SNP rs609539 and SEQ ID NO: 8 with SNP rs7975838 and SEQ ID NO: 9 with SNP rs12063296 and SEQ ID NO: 10 with SNP rs16913719; or SEQ ID NO: 11 with SNP rs11497898 and SEQ ID NO: 12 with SNP rs17168572 and SEQ ID NO: 13 with SNP rs16933412 and SEQ ID NO: 14 with SNP rs16864505 and SEQ ID NO: 1 with SNP rs666247 and SEQ ID NO: 2 with SNP rs12707034 and SEQ ID NO: 3 with SNP rs707497 and SEQ ID NO: 4 with SNP rs17024172 and SEQ ID NO: 5 with SNP rs16950705 and SEQ ID NO: 6 with SNP rs11956461 etc. Further envisaged are all other 10 SNP permutations or groupings of the mentioned SNPs.

Further preferred are combinations or panels of any possible 11 of the SNPs of the present invention, e.g. SEQ ID NO: 1 with SNP rs666247 and SEQ ID NO: 2 with SNP rs12707034 and SEQ ID NO: 3 with SNP rs707497 and SEQ ID NO: 4 with SNP rs17024172 and SEQ ID NO: 5 with SNP rs16950705 and SEQ ID NO: 6 with SNP rs11956461 and SEQ ID NO: 7 with SNP rs609539 and SEQ ID NO: 8 with SNP rs7975838 and SEQ ID NO: 9 with SNP rs12063296 and SEQ ID NO: 10 with SNP rs16913719 and SEQ ID NO: 11 with SNP rs11497898, or SEQ ID NO: 12 with SNP rs17168572 and SEQ ID NO: 13 with SNP rs16933412 and SEQ ID NO: 14 with SNP rs16864505 and SEQ ID NO: 1 with SNP rs666247 and SEQ ID NO: 2 with SNP rs12707034 and SEQ ID NO: 3 with SNP rs707497 and SEQ ID NO: 4 with SNP rs17024172 and SEQ ID NO: 5 with SNP rs16950705 and SEQ ID NO: 6 with SNP rs11956461 and SEQ ID NO: 7 with SNP rs609539 and SEQ ID NO: 8 with SNP rs7975838 etc. Further envisaged are all other 11 SNP permutations or groupings of the mentioned SNPs.

Further preferred are combinations or panels of any possible 12 of the SNPs of the present invention, e.g. SEQ ID NO: 1 with SNP rs666247 and SEQ ID NO: 2 with SNP rs12707034 and SEQ ID NO: 3 with SNP rs707497 and SEQ ID NO: 4 with SNP rs17024172 and SEQ ID NO: 5 with SNP rs16950705 and SEQ ID NO: 6 with SNP rs11956461 and SEQ ID NO: 7 with SNP rs609539 and SEQ ID NO: 8 with SNP rs7975838 and SEQ ID NO: 9 with SNP rs12063296 and SEQ ID NO: 10 with SNP rs16913719 and SEQ ID NO: 11 with SNP rs11497898 and SEQ ID NO: 12 with SNP rs17168572; or SEQ ID NO: 13 with SNP rs16933412 and SEQ ID NO: 14 with SNP rs16864505 and SEQ ID NO: 1 with SNP rs666247 and SEQ ID NO: 2 with SNP rs12707034 and SEQ ID NO: 3 with SNP rs707497 and SEQ ID NO: 4 with SNP rs17024172 and SEQ ID NO: 5 with SNP rs16950705 and SEQ ID NO: 6 with SNP rs11956461 and SEQ ID NO: 7 with SNP rs609539 and SEQ ID NO: 8 with SNP rs7975838 and SEQ ID NO: 9 with SNP rs12063296 and SEQ ID NO: 10 with SNP rs16913719 etc. Further envisaged are all other 12 SNP permutations or groupings of the mentioned SNPs.

Further preferred are combinations or panels of any possible 13 of the SNPs of the present invention, e.g. SEQ ID NO: 1 with SNP rs666247 and SEQ ID NO: 2 with SNP rs12707034 and SEQ ID NO: 3 with SNP rs707497 and SEQ ID NO: 4 with SNP rs17024172 and SEQ ID NO: 5 with SNP rs16950705 and SEQ ID NO: 6 with SNP rs11956461 and SEQ ID NO: 7 with SNP rs609539 and SEQ ID NO: 8 with SNP rs7975838 and SEQ ID NO: 9 with SNP rs12063296 and SEQ ID NO: 10 with SNP rs16913719 and SEQ ID NO: 11 with SNP rs11497898 and SEQ ID NO: 12 with SNP rs17168572 and SEQ ID NO: 13; or SNP rs16933412 and SEQ ID NO: 14 with SNP rs16864505 and SEQ ID NO: 1 with SNP rs666247 and SEQ ID NO: 2 with SNP rs12707034 and SEQ ID NO: 3 with SNP rs707497 and SEQ ID NO: 4 with SNP rs17024172 and SEQ ID NO: 5 with SNP rs16950705 and SEQ ID NO: 6 with SNP rs11956461 and SEQ ID NO: 7 with SNP rs609539 and SEQ ID NO: 8 with SNP rs7975838 and SEQ ID NO: 9 with SNP rs12063296 and SEQ ID NO: 10 with SNP rs16913719 and SEQ ID NO: 11 with SNP rs11497898 and SEQ ID NO: 12 with SNP rs17168572 etc. Further envisaged are all other 13 SNP permutations or groupings of the mentioned SNPs.

Further preferred is a combination or panel of all 14 SNPs of the present invention, i.e. SEQ ID NO: 1 with SNP rs666247 and SEQ ID NO: 2 with SNP rs12707034 and SEQ ID NO: 3 with SNP rs707497 and SEQ ID NO: 4 with SNP rs17024172 and SEQ ID NO: 5 with SNP rs16950705 and SEQ ID NO: 6 with SNP rs11956461 and SEQ ID NO: 7 with SNP rs609539 and SEQ ID NO: 8 with SNP rs7975838 and SEQ ID NO: 9 with SNP rs12063296 and SEQ ID NO: 10 with SNP rs16913719 and SEQ ID NO: 11 with SNP rs11497898 and SEQ ID NO: 12 with SNP rs17168572 and SEQ ID NO: 13 with SNP rs16933412 and SEQ ID NO: 14 with SNP rs16864505.

In yet another preferred embodiment the present invention relates the isolated nucleic acid or group or panel of nucleic acids, and/or corresponding SNPs as marker for beta thalassemia, wherein said panel or group comprises at least:

(i) SEQ ID NO: 1 except for a single polymorphic change at position 501, where wildtype nucleotide T is replaced by indicator nucleotide C; and/or

(ii) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and/or

(iii) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and/or

(iv) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and/or

(v) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 5 except for a single polymorphic change at position 501, where wildtype nucleotide C is replaced by indicator nucleotide T; and/or

(vi) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 5 except for a single polymorphic change at position 501, where wildtype nucleotide C is replaced by indicator nucleotide T; and SEQ ID NO: 6 except for a single polymorphic change at position 501, where wildtype nucleotide C is replaced by indicator nucleotide T; and/or

(vii) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 5 except for a single polymorphic change at position 501, where wildtype nucleotide C is replaced by indicator nucleotide T; and SEQ ID NO: 6 except for a single polymorphic change at position 501, where wildtype nucleotide C is replaced by indicator nucleotide T; and SEQ ID NO: 7 except for a single polymorphic change at position 501, where wildtype nucleotide G is replaced by indicator nucleotide A; and/or

(viii) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 5 except for a single polymorphic change at position 501, where wildtype nucleotide C is replaced by indicator nucleotide T; and SEQ ID NO: 6 except for a single polymorphic change at position 501, where wildtype nucleotide C is replaced by indicator nucleotide T; and SEQ ID NO: 7 except for a single polymorphic change at position 501, where wildtype nucleotide G is replaced by indicator nucleotide A; and SEQ ID NO: 8 except for a single polymorphic change at position 501, where wildtype nucleotide T is replaced by indicator nucleotide C; and SEQ ID NO: 9 except for a single polymorphic change at position 501, where wildtype nucleotide A is replaced by indicator nucleotide G; and SEQ ID NO: 10 except for a single polymorphic change at position 501, where wildtype nucleotide C is replaced by indicator nucleotide T; and SEQ ID NO: 11 except for a single polymorphic change at position 501, where wildtype nucleotide T is replaced by indicator nucleotide C; and SEQ ID NO: 12 except for a single polymorphic change at position 501, where wildtype nucleotide A is replaced by indicator nucleotide G; and SEQ ID NO: 13 except for a single polymorphic change at position 501, where wildtype nucleotide T is replaced by indicator nucleotide C; and SEQ ID NO: 14 except for a single polymorphic change at position 501, where wildtype nucleotide C is replaced by indicator nucleotide T; and/or

(ix) SEQ ID NO: 8 except for a single polymorphic change at position 501, where wildtype nucleotide T is replaced by indicator nucleotide C; and SEQ ID NO: 14 except for a single polymorphic change at position 501, where wildtype nucleotide C is replaced by indicator nucleotide T; and/or

(x) SEQ ID NO: 8 except for a single polymorphic change at position 501, where wildtype nucleotide T is replaced by indicator nucleotide C; and SEQ ID NO: 9 except for a single polymorphic change at position 501, where wildtype nucleotide A is replaced by indicator nucleotide G; and/or

(xi) SEQ ID NO: 2 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 13 except for a single polymorphic change at position 501, where wildtype nucleotide T is replaced by indicator nucleotide C; and/or any one of herein above defined panels or combinations of SNPs. Particularly preferred is the group of (i), (ii), (viii), (ix), (x), and (xi).

In specific embodiments of the present invention any of the above mentioned panels or groups or combinations of SNPs may further be combined with additional markers, e.g. SNPs of haplotypes or haplogroups on the same chromosome as defined herein, e.g. in Example 2 or in Table 6, or in FIG. 6, or in the above mentioned group of SNPs included in the haplotypes. The panels or groups or combinations of SNPs may also be combined with independent marker, e.g. phenotypic markers such as blood related marker, e.g. a subject's blood volume, the fraction of hemoglobin A2, the fraction of hemoglobin A, genomic sequence information, or other suitable markers of beta thalassemia known to the person skilled in the art.

In further specific embodiments of the present invention the herein above defined panels, in particular groups (i) to (xi), may show the indicator nucleotide at one or two alleles of a single subject.

In a further aspect the present invention relates to a method for detecting or diagnosing beta thalassemia in a subject comprising the steps of:

(c) isolating a nucleic acid from a subject's sample

(d) determining the nucleotide sequence and/or molecular structure present at one or more polymorphic sites as defined herein above;

wherein the presence of an indicator nucleotide as defined herein above is indicative of the presence of beta thalassemia.

The term “detecting beta thalassemia” as used herein means that the presence of beta thalassemia may be determined in a human being. The term also includes the detection or identification of a beta thalassemia carrier status, which may be phenotypically unapparent or be associated with symptoms like mild anemia etc.

The term “diagnosing beta thalassemia” as used herein means that beta thalassemia may be identified in a human being. The term in particular refers to the identification of situations in which the subject is actually afflicted by disease symptoms or shows the phenotype of the diseases, i.e. is for example afflicted by anemia.

The term “determining the nucleotide sequence at a polymorphic site” as used herein refers to any suitable method or technique of detecting the identity of the nucleotide at position 501 of any one of or any grouping or panel comprising SEQ ID NO: 1 to 14. This determination method may predominantly be a sequencing technique or a technique based on complementary nucleic acid binding.

The term “determining the molecular structure present at a polymorphic site” as used herein refers to an alternative method of detecting the identity of the nucleotide at position 501 of any one of or any grouping or panel comprising SEQ ID NO: 1 to 14, e.g. via structural or 3 dimensional properties of the nucleic acid etc.

Upon the determination of the identity of the nucleotide at position 501 of any one of or any grouping or panel comprising SEQ ID NO: 1 to 14, different scenarios may be encountered, the most typical ones being:

(a) the analyzed position 501 of any one of SEQ ID NO: 1 to 14 shows the wildtype nucleotide as defined herein above; in this case the presence of beta thalassemia may be excluded. Any possible further symptoms may accordingly be attributed to a different disease or disorder, e.g. a different anemia.

(b) the analyzed position 501 of some of the SEQ ID NO: 1 to 14, e.g. in one of the above defined panels, shows the wildtype nucleotide, whereas one or more than one, or possibly all of SEQ ID NO: 1 to 14 show an indicator nucleotide at the position; in this case the presence of beta thalassemia may be given.

(c) the analyzed position 501 of some of the SEQ ID NO: 1 to 14, e.g. in one of the above defined panels, shows in one or more than one, or possibly all of SEQ ID NO: 1 to 14 an indicator nucleotide at the position, whereas in other panel members a wildtype nucleotide or a nucleotide not identical neither to the wildtype nor the indicator nucleotide is present; in this case also the presence of beta thalassemia may be given.

(d) the analyzed position 501 of any one of SEQ ID NO: 1 to 14 shows a nucleotide which is not the indicator nucleotide as defined herein above; this nucleotide may a different nucleotide not identical to the wildtype nucleotide, but also not identical to the indicator nucleotide as define herein; in this case the presence of beta thalassemia cannot be excluded. Any possible further symptoms may accordingly be taken into account. Also additional detection steps, further genetic analysis etc. may be necessary in order to determine the subject's health state, i.e. in order to confirm that the subject is indeed afflicted by beta thalassemia.

(e) the analyzed position 501 of some of SEQ ID NO: 1 to 14 shows a nucleotide which is not the indicator nucleotide as defined herein above; this nucleotide may a different nucleotide not identical to the wildtype nucleotide, but also not identical to the indicator nucleotide as define herein, whereas in other panel members a wildtype nucleotide is present; also in this case the presence of beta thalassemia cannot be excluded. Any possible further symptoms may accordingly be taken into account. Also additional detection steps, further genetic analysis etc. may be necessary in order to determine the subject's health state, i.e. in order to confirm that the subject is indeed afflicted by beta thalassemia.

According to the above scenarios (a) to (e) the presence of presence of beta thalassemia may determined. In specific embodiments, the test may be repeated, e.g. 1, 2, 3, 4 or 5 or more often. Furthermore, upon an unclear or inconclusive result, e.g. based on the use of only one of the presently described SNPs, an enlarged panel of SNPs may be analyzed, e.g. the group of SNPs to be analyzed may be increased by 1, 2, 3, 4, 5, 6, 7, 10 etc.

In a further particularly preferred embodiment the above described method is a method for detecting or diagnosing beta thalassemia minor in a subject. Accordingly, the detection of scenarios (a) to (e) as defined herein above may be associated with the presence of thalassemia minor in subject, or the absence of this disease and/or the possible presence of a different, similar disorder, e.g. a different anemia.

A “subject's sample” as used herein may be any sample derived from any suitable part or portion of a subject's body. The sample may, in one embodiment, be derived from pure tissues or organs or cell types, or derived from very specific locations, e.g. comprising only one type of tissue, cell, or organ. In further embodiments, the sample may be derived from mixtures of tissues, organs, cells, or from fragments thereof. Samples may, for example, be obtained from organs or tissues such as the gastrointestinal tract, the vagina, the stomach, the heart, the tongue, the pancreas, the liver, the lungs, the kidneys, the skin, the spleen, the ovary, a muscle, a joint, the brain, the prostate, the lymphatic system or organ or tissue known to the person skilled in the art. In further embodiments of the invention the sample may be derived from body fluids, e.g. from blood, serum, saliva, urine, stool, ejaculate, lymphatic fluid etc. In a particularly preferred embodiment of the present invention the above mentioned sample is be a mixture of tissues, organs, cells and/or fragments thereof, or a tissue or organ specific sample, such as a tissue biopsy from vaginal tissue, tongue, pancreas, liver, spleen, ovary, muscle, joint tissue, neural tissue, gastrointestinal tissue, tumor tissue, or a body fluid, blood, serum, saliva, or urine. Preferred is the use of blood sample. Particularly preferred is the employment of blood samples comprising DNA-containing cells, e.g. non-matured red blood cells, erythrocyte precursor cells, leukocytes etc. Also envisaged is the use of bone marrow cells, erythropoietic cells etc. The sample used in the context of the present invention should preferably be collected in a clinically acceptable manner, more preferably in a way that nucleic acids or proteins are preserved.

In specific embodiments blood samples may be used for different types of analysis, e.g. DNA-based SNP analysis as well as the analysis of blood components, the concentration of hemoglobin chains etc,

In further embodiments of the present invention the sample may contain one or more than one cell, e.g. a group of histologically or morphologically identical or similar cells, or a mixture of histologically or morphologically different cells. Preferred is the use of histologically identical or similar cells, e.g. stemming from one confined region of the body.

In a specific embodiment a sample may be obtained from the same subject at different points in time, obtained from different organs or tissues of the same subject, or form different organs or tissues of the same subject at different points in time. For example, a sample of specific tissue and of one or more samples of a neighbouring region of the same tissue or organ may be taken.

In further preferred embodiment of the present invention the mentioned determination of the nucleotide sequence may be carried out through allele-specific oligonucleotide (ASO)-dot blot analysis, primer extension assays, iPLEX SNP genotyping, Dynamic allele-specific hybridization (DASH) genotyping, the use of molecular beacons, tetra primer ARMS PCR, a flap endonuclease invader assay, an oligonucleotide ligase assay, PCR-single strand conformation polymorphism (SSCP) analysis, quantitative real-time PCR assay, SNP microarray based analysis, restriction enzyme fragment length polymorphism (RFLP) analysis, targeted resequencing analysis and/or whole genome sequencing analysis.

The term “allele-specific oligonucleotide (ASO)-dot blot analysis” as used herein refers to the employment of a short piece of synthetic DNA, which is typically complementary to the sequence of a polymorphic target site, in dot blot assay or, alternatively, in a Southern blot assay. The alleles specific oligonucleotide may be an oligonucleotide of 15-21 bases in length, e.g. spanning 15 to 21 nucleotides around position 501 of SEQ ID NO: 1 to 14 or the complementary sequence thereof. The ASO may vary in length, may be chose from either of the two nucleic acid strands and the specificity of its binding in the dot blot or Southern blot may be modified by suitable buffer, hybridizing or washing conditions, which would be known to the person skilled in the art. The ASO may be labeled with any suitable label, e.g. with a radioactive, enzymatic, or fluorescent tag.

The term “primer extension assay” as used herein refers to a two step process that first involves the hybridization of a probe to the bases immediately upstream of the polymorphic nucleotide followed by a mini-sequencing reaction, in which DNA polymerase extends the hybridized primer by adding a base that is complementary to the polymorphic nucleotide. The incorporated base may subsequently be detected and can thus determine the SNP allele. The primer extension method may be used in the context of further assay formats, e.g. detection techniques including MALDI-TOF Mass spectrometry and ELISA-like methods.

The term “iPLEX SNP genotyping” as used herein refers to a method involving the use of a MassARRAY mass spectrometer and extension probes designed in such a way that 40 different SNP assays can be amplified and analyzed in a PCR cocktail. The extension reaction preferably uses ddNTPs and the detection of the SNP allele is typically dependent on the actual mass of the extension product. Further details are known to the person skilled in the art.

The term “Dynamic allele-specific hybridization (DASH) genotyping” refers to a technique taking advantage of the differences in the melting temperature in DNA that results from the instability of mismatched base pairs. Preferably, in a first step, a genomic segment may be amplified and attached to a bead through a PCR reaction, e.g. with a biotinylated primer. In the second step, the amplified product may be attached to a streptavidin column and washed, e.g. preferably with NaOH, to remove the unbiotinylated strand. Subsequently, an allele-specific oligonucleotide may be added in the presence of a molecule that fluoresces when bound to double-stranded DNA. The intensity may subsequently be measured as temperature is increased until the Tm can be determined. A SNP will typically result in a lower than expected Tm. In a preferred embodiment the process may be carried out on an automated basis.

The term “use of molecular beacons” as used herein refers to a detection of a polymorphism by using specifically designed single-stranded oligonucleotide probe comprising complementary regions at each end and a probe sequence located in between. This design typically allows the probe to take on a hairpin structure or stem-loop structure. The probe may preferably comprise at one end a fluorophore and at the other end a fluorescence quencher and, in certain embodiments, be engineered such that only the probe sequence is complementary to the genomic DNA that will be used in the assay. If the probe sequence of the molecular beacon encounters its target DNA, it will anneal, hybridize and fluoresce. If, however, the probe sequence encounters a modified target sequence with a non-complementary nucleotide, the molecular beacon may preferably stay in its natural hairpin state and no fluorescence may be observed, thereby allowing a distinction between a wildtype situation and modification thereof. In preferred embodiments of the invention more than one such molecular beacon may be used, e.g. one for a wildtype sequence, and a further one for a sequence including an indicator nucleotide. Thereby the presence of at least the wildtype nucleotide and the indicator nucleotide may be determined.

The term “tetra primer ARMS PCR” as used herein refers to the method involving two pairs of primers to amplify two alleles in one PCR reaction. The primers are typically designed such that the two primer pairs overlap at a polymorphic site or SNP location but each match perfectly to only one of the possible SNPs. As a result, if a given allele is present in the PCR reaction, the primer may pair specific to that allele and may subsequently produce a product but not to the alternative allele with a different SNP. The two primer pairs may, in further embodiments, also designed such that their PCR products are of a significantly different length allowing, for example, for easily distinguishable bands by gel electrophoresis.

The term “flap endonuclease invader assay” as used herein refers to the use of a flap endonuclease cleavase which is combined with two specific oligonucleotide probes that, together with the target DNA, can form a tripartite structure recognized by the cleavase The first probe, i.e. the invader oligonucleotide is preferably complementary to the 3′ end of the target DNA. The last base of the invader oligonucleotide may be a non-matching base that overlaps the SNP nucleotide in the target DNA. The second probe may be an allele-specific probe which is complementary to the 5′ end of the target DNA, but may also extend past the 3′ side of the SNP nucleotide. The allele-specific probe may contain a base complementary to the SNP nucleotide. If the target DNA contains the desired allele, the invader and allele-specific probes may bind to the target DNA forming the tripartite structure. The cleavase may subsequently cleave and release the 3′ end of the allele-specific probe. In preferred embodiments, the invader assay may be coupled with a fluorescence resonance energy transfer (FRET) system to detect the cleavage event.

The term “quantitative real-time PCR assay” as used herein refers to an assay preferably performed with a Taqman enzyme or a similar activity, concurrently with a PCR reaction, wherein the results can be read in real-time as the PCR reaction proceeds. The assay typically requires forward and reverse PCR primers that will amplify a region that includes the polymorphic site, preferably primers binding in the 5′ or 3′ region with respect to position 501 of any one of SEQ ID NO: 1 to 14. Allele discrimination may, in specific embodiments also be achieved using FRET combined with one or two allele-specific probes that hybridize to the SNP polymorphic site. The probes may have a fluorophore linked to their 5′ end and a quencher molecule linked to their 3′ end. While the probe is intact, the quencher may remain in close proximity to the fluorophore, eliminating the fluorophore's signal. During the PCR amplification step, if the allele-specific probe is perfectly complementary to the SNP allele, it may bind to the target DNA strand and then get degraded by 5′-nuclease activity of the Taq polymerase as it extends the DNA from the PCR primers. If the allele-specific probe is not perfectly complementary, it may have a lower melting temperature and not bind as efficiently.

The term “oligonucleotide ligase assay” as used herein refers to an enzymatic reaction catalyzed by DNA ligase which may be used to interrogate a SNP by hybridizing two probes directly over the SNP polymorphic site, whereby ligation can occur if the probes are identical to the target DNA. Typically, two probes are designed: an allele-specific probe which hybridizes to the target DNA so that its 3′ base is situated directly over the SNP nucleotide and a second probe that hybridizes the template upstream (downstream in the complementary strand) of the SNP polymorphic site providing a 5′ end for the ligation reaction. Ligated or unligated products may subsequently be detected by gel electrophoresis, MALDI-TOF mass spectrometry or by capillary electrophoresis.

The term “PCR-single strand conformation polymorphism (SSCP) analysis” as used herein refers a method, capable of identifying sequence variations in a single strand of DNA, typically between 150 and 250 nucleotides in length. The method is based on the fact that single-stranded DNA (ssDNA) folds into a tertiary structure. The conformation is typically sequence dependent and most single base pair mutations will alter the shape of the structure. When applied to a gel, the tertiary shape may determine the mobility of the ssDNA, which provides a mechanism to differentiate between polymorphic alleles. In preferred embodiments the method first involves a PCR amplification of a target DNA. The double-stranded PCR products may be denatured using heat and formaldehyde to produce ssDNA. The ssDNA may be applied to a non-denaturing electrophoresis gel and allowed to fold into a tertiary structure.

The term “SNP microarray based analysis” as used herein refers to the employment of high-density oligonucleotide SNP arrays comprising, for example, 100, 1000, or more than 10000 probes arrayed on a chip, allowing for many SNPs to be interrogated simultaneously. Target DNA may be hybridized to the array, preferably by using several redundant probes to interrogate each SNP. In specific embodiments, probes may be designed to have the SNP site in several different locations as well as containing mismatches to the SNP allele. In further embodiments, the differential amount of hybridization of the target DNA to each of these redundant probes may also allow the determination of homozygous and heterozygous alleles, e.g. to detect whether one or two of the alleles of SEQ ID NO: 1 to 14 show the indicator sequence or not. An example of a SNP microarrays, which is also envisaged by the present invention is the Affymetrix Human SNP 5.0 GeneChip.

The term “restriction enzyme fragment length polymorphism (RFLP) analysis” used herein refers to the performance of a digestion on a genomic sample and the determination of fragment lengths, e.g. through a gel assay, allowing to ascertain whether or not the enzymes cut at expected restriction sites. The RFLP analysis may preferably be carried out on the basis of PCR amplified fragments around position 501 of any one of SEQ ID NO: 1 to 14. The corresponding primer binding sites and the length may be determined in dependence on the availability of suitable restriction sites.

The term “targeted resequencing analysis” as used herein refers to capturing and sequencing of the regions of interest, wherein the capturing may be in solution or on an array and the sequencing can be performed by any first, second or third generation sequencing platform. Further details and features would be known to the person skilled in the art.

The term “whole genome sequencing analysis” as used herein refers to the determination of the sequence of the entire genome of a subject, preferably of both alleles of a polymorphic site based on high-throughput sequencing technology, e.g. Next-generation sequencing technologies such as pyrosequencing. The techniques may, in certain embodiments, also be used for the sequencing of portions of the genomic, e.g. small regions of interest. This technique may, preferred embodiments, also be used for the determination of haplotypes or haplogroups in chromosomic regions or on specific chromosomes.

In further embodiments of the present invention a method for detecting or diagnosing beta thalassemia may comprises one or more additional steps relating to the determination of blood structure, blood volume, blood components, the presence or concentration of blood components or factors, the determination of blood parameters, the determination of blood compound concentration, the determination of hemoglobin concentration or behavior etc. These steps may comprise taking a sample from a subject, or analyzing a sample previously taken from a subject. The above mentioned steps may in particular also include a comparison with standards or values associated with a healthy state as would be known to the person skilled in the art.

Particularly preferred is the determination of the Hb A2 concentration in a sample. For the determination of the Hb A2 concentration any suitable method or approach known to the person skilled in the art may be used.

Preferably, the determination of Hb A2 concentration may be carried out via HPLC, microchromatography, isoelectric focusing, or capillary electrophoresis, or any mixture thereof, or any other suitable method not yet known. Furthermore, a result obtained with one approach may preferably be confirmed with another method. Particularly preferred is the use of a catio-exchange HPLC, which allows a quantitative and qualitative hemoglobin analysis, leading to an effective measurement of the Hb A2 concentration.

If, upon the determination of the Hb A2 concentration, an Hb A2 value of about 2% to 3.2% is obtained, the subject may be considered being in a healthy state. Preferably, this concentration may indicate that the subject is not afflicted by beta thalassemia and that the subject is not a beta thalassemia carrier.

If, upon the determination of the Hb A2 concentration, an Hb A2 value of more than about 3.2%, e.g. about 3.3%, 3.4% 3.5%, 3.6%, 3.7% or 3.8% to about to 7% is obtained, the subject may be considered being affected by beta thalassemia. Furthermore, this concentration may that the subject is a beta thalassemia carrier.

The Hb A2 value may in specific embodiments be used to modify results obtained by the SNP analysis, e.g. if polymorphisms are encountered not falling within the group of wildtype or indicator SNPs, or to corroborate results if in a larger panel only very few SNPs show indicator state, whereas the majority shows wildtype state.

In further preferred embodiments, the present invention the method as mentioned herein above may be combined with molecular functional analysis steps. For example, in cases in which the SNPs could be shown to be associated with specific molecular pattern, the corresponding molecular pattern may additionally be analyzed in order to improve the diagnostic value of the method. The term “molecular pattern” as used herein refers to any suitable molecular or functional state, e.g. functional genomic state, which is linked to one or more of the SNPs of the present invention.

In a particularly preferred embodiment of the present invention a method as described herein above may comprise the determination of the nucleotide sequence and/or molecular structure present at polymorphic sites of SEQ ID NO: 8 and SEQ ID NO: 9 in combination with the detection of a DNAse hypersensitivity site in the genomic vicinity of SEQ ID NO: 8, in the genomic vicinity of SEQ ID NO: 9 or in the genomic vicinity of SEQ ID NO: 8 and SEQ ID NO: 9. The term “genomic vicinity” as used in the context of this embodiment refers to regions of about 0.75 kb, 1 kb, 1.5 kb, 2 kb, 2.5 kb, 3 kb, 4 kb, 5 kb or more or any value in between of the region indicated herein above with respect to the genomic localization of the sequence of SEQ ID NO: 8 or SEQ ID NO: 9.

The term “DNAse hypersensitivity site” as used herein refers to a short region of chromatin in which the nucleosomal structure of the genome may not be organized in the usual fashion, which may results in a significant increase in sensitivity to an enzyme attack than in bulk chromatin. Preferably, such a DNAse hypersensitivity site may be detected by its super sensitivity to cleavage by DNase I and/or other nucleases such as DNase II or micrococcal nucleases.

In specific embodiments of the present invention DNase I, DNase II and/or micrococcal nuclease or any other suitable enzyme known to the person skilled in the art may accordingly be used for the analysis of genomic DNA obtained from a subject, e.g. derived from a sample as described herein above.

In the presence of an indicator nucleotide within the SNPs associated with SEQ ID NO: 8, or with SEQ ID NO: 9, or with SEQ ID NO: 8 and SEQ ID NO: 9 as defined herein above and in the presence of a DNAse hypersensitivity site in the vicinity of the corresponding SNP, i.e. in the genomic vicinity of SEQ ID NO: 8, in the genomic vicinity of SEQ ID NO: 9, or in the genomic vicinity of SEQ ID NO: 8 and 9, as mentioned above, a subject may be considered to be afflicted by beta thalassemia.

In further, specific embodiments the present invention relates to a pharmaceutical composition comprising a compound which is able to compensate, reduce or reverse the DNAse hypersensitivity in the genomic vicinity of SEQ ID NO: 8, in the genomic vicinity of SEQ ID NO: 9, or in the in the genomic vicinity of SEQ ID NO: 8 and SEQ ID NO: 9. In a particular embodiment said pharmaceutical composition may be for use in the treatment of beta thalassemia.

In a further particularly preferred embodiment of the present invention a method as described herein above may comprise the determination of the nucleotide sequence and/or molecular structure present at polymorphic sites of SEQ ID NO: 2 and SEQ ID NO: 4 and SEQ ID NO: 13 in combination with the detection of a histone 3 lysine 27 trimethylation in the genomic vicinity of SEQ ID NO: 2 and/or SEQ ID NO: 4 and/or SEQ ID NO: 13. The term “genomic vicinity” as used in the context of this embodiment refers to regions of about 0.6 kb, 0.7 kb, 0.75 kb, 0.8 kb, 0.9 kb, 1 kb, 1.25 kb, 1.5 kb, 1.75 kb, 2 kb, 2.5 kb, 3 kb, 3.5 kb, 4 kb or more or any value in between of the region indicated herein above with respect to the genomic localization of the sequence of SEQ ID NO: 2 or SEQ ID NO: 4 or SEQ ID NO: 13.

The term “histone 3 lysine 27 trimethylation” refers to the addition of methyl residues to lysine 27 of histone 3 molecules within human genomic DNA. The methylation may be carried by a histone methyltranferase. Typically, a trimethylation at histone 3 lysine 27 is assumed to act as repressive mark.

In specific embodiments of the present invention histone 3 lysine 27 methylation specific detection systems, e.g. specific antibodies etc., may be used in order to detect the presence of histone 3 lysine 27 trimethylation in the genomic vicinity of the sequence of SEQ ID NO: 2 or SEQ ID NO: 4 or SEQ ID NO: 13. For example, genomic DNA obtained from a subject's sample may be directly used upon an specific enrichment step for the detection of histone 3 lysine 27 trimethylation. Suitable methods and further details would be known to the person skilled in the art.

In the presence of an indicator nucleotide within the SNPs associated with SEQ ID NO: 2, or with SEQ ID NO: 4, or with SEQ ID NO: 13, or with SEQ ID NO: 2 and SEQ ID NO:4 and SEQ ID NO:13, or SEQ ID NO:2 and SEQ ID NO:4, or SEQ ID NO:4 and SEQ ID NO:13, or SEQ ID NO:2 and SEQ ID NO:13 as defined herein above and in the presence of histone 3 lysine 27 trimethylation in the vicinity of the corresponding SNP, i.e. in the genomic vicinity of SEQ ID NO: 2, in the genomic vicinity of SEQ ID NO: 4, or in the genomic vicinity of SEQ ID NO: 13 etc., as mentioned above, a subject may be considered to be afflicted by beta thalassemia.

In further, specific embodiments the present invention relates to a pharmaceutical composition comprising a compound which is able to compensate, reduce or reverse the histone 3 lysine 27 trimethylation in the vicinity of the corresponding SNP of SEQ ID NO: 2, in the genomic vicinity of SEQ ID NO: 4, or in the in the genomic vicinity of SEQ ID NO: 13 etc. In a particular embodiment said pharmaceutical composition may be for use in the treatment of beta thalassemia.

In further specific embodiments further SNPs of the present invention, e.g. SNPs associated with SEQ ID NO: 1, 3, 5, 6, 7, 10, 11, 12, or 14 as defined herein above, which show no obvious functional relationship to a gene or regulatory region in the vicinity of said SNP may have a functional relation with respect to noncoding RNAs (Nardella C. et al., Curr Top Microbiol Immunol. 2010; 347:135-68). Corresponding noncoding RNAs may accordingly be detected with the help of suitable methods known to the person skilled in the art. In further embodiments, such noncoding RNAs as well as repressor or activator factors thereof may be used for an improved diagnostic approach for the detection of beta thalassemia, or for a corresponding therapeutic approach.

In another aspect the present invention relates to a composition for detecting or diagnosing beta thalassemia in a subject comprising a nucleic acid affinity ligand for one or more polymorphic sites as defined herein above. In a preferred embodiment the present invention relates to such a composition for detecting or diagnosing beta thalassemia minor as defined herein above.

The term “nucleic acid affinity ligand” as used herein refers to a nucleic acid molecule being able to bind to a polymorphic sites as defined above. Preferably, the affinity ligand is able to bind the sequence of SEQ ID NO: 1 to 14, or fragments thereof, which comprise the polymorphic site as defined herein above, wherein said sequence of SEQ ID NO: 1 to 14 comprise the respective indicator nucleotide as described herein above. In further embodiments of the present invention the nucleic acid affinity ligand may also be able to specifically bind to a DNA sequence being at least 90%, 91%, 92%, 93%, 94%, 95%, 96%, 97%, 98% or 99% or 99.5% or 99.6%, 99.7%, 99.8%, or 99.9% identical to the sequence of SEQ ID NO: 1 to 14, or fragments thereof, which comprise the polymorphic site as defined herein above, wherein said sequence of SEQ ID NO: 1 to 14 comprises the respective indicator nucleotide as described herein above, or to any fragments of said sequences. In further embodiments of the present invention an nucleic acid affinity ligand according to the present invention may also be able to specifically bind to a DNA sequences of SEQ ID NO: 1 to 14, which comprise the polymorphic site as defined herein above, i.e. to wildtype sequences which do not comprise the respective indicator nucleotide as described herein above. In further embodiments of the present invention the nucleic acid affinity ligand may also be able to specifically bind to a DNA sequence being at least 90%, 91%, 92%, 93%, 94%, 95%, 96%, 97%, 98% or 99% or 99.5% or 99.6%, 99.7%, 99.8%, or 99.9% identical to the sequence of SEQ ID NO: 1 to 14 which comprises the polymorphic site as defined herein above, i.e. to wildtype sequences which do not comprise the respective indicator nucleotide as described herein above or to fragments thereof. In even further embodiments, the present invention relates to nucleic acid affinity ligands binding a sequence complementary to the sequence of SEQ ID NO: 1 to 14, which comprises the polymorphic site as defined herein above an indicator nucleotide as defined herein above, which may comprise or may not comprise the indicator nucleotide.

In further specific embodiments said nucleic acid affinity ligand may be a short nucleic acid molecule, e.g. a RNA, DNA, PNA, CNA, HNA, LNA or ANA molecule or any other suitable nucleic acid format known to the person skilled in the art, being capable of specifically binding to the sequence of SNPs (e.g. indicator or wildtype sequence) of SEQ ID NO: 1 to 14.

In further specific embodiments said nucleic acid affinity ligand may comprise any suitable functional component known to the skilled person, e.g. a tag, a fluorescent label, a radioactive label, a dye, a binding or recognition site for a protein or antibody or peptide, a further stretch of DNA useful for PCR approaches, a stretch of DNA useful as recognition site for restriction enzymes etc. The nucleic acid affinity ligand may further be provided in the form of a catalytic RNA specifically binding to and cleaving a sequence comprising the SNP according to the present invention, e.g. either the indicator nucleotide or the wildtype nucleotide or a different nucleotide at the polymorphic site.

In further embodiments the present invention envisages pairs of nucleic acid affinity ligand of which one is able to specifically bind to the wildtype sequence of SEQ ID NO: 1 to 14, and the other is specifically able to bind to the sequence of SEQ ID NO: 1 to 14 including the indicator nucleotide as defined herein above. Such pairs may further be distinguished by, for example, differential labels, different dyes, or any other different functionality as described herein. Furthermore, more than two pairs, e.g. for each of SEQ ID NO: 1 to 14 a pair or a sub-group thereof may be provided, which are also distinguished by differential dyes, labels, or other functionalities.

In further specific embodiments the present invention also relates to non-nucleic acid affinity ligands specific for one or more polymorphic sites as defined herein above. Such affinity ligands may be peptides, aptamer like elements, antibodies, DNA motif recognizing proteins, e.g. restriction enzymes, or combinations of these with nucleic acids.

The composition according to the present invention may additionally comprise further ingredients necessary or useful for the detection of beta thalassemia, such as buffers, dNTPs, a polymerase, ions like bivalent cations or monovalent cations, hybridization solutions etc.

In yet another preferred embodiment of the present invention the affinity ligand as mentioned herein above may be an oligonucleotide specific for one or more polymorphic sites as defined herein above, or a probe specific for one or more polymorphic sites as defined herein above. The term “oligonucleotide specific for one or more polymorphic sites” as used herein refers to a nucleic acid molecule, preferably a DNA molecule of a length of about 12 to 38 nucleotides, preferably of about 15 to 30 nucleotides. The oligonucleotide may have, for example, a length of 16, 17, 18, 19, 20, 21, 22, 23, 24, 25, 26, 27, 28, 29, or 30 nucleotides. These molecules may preferably be complementary to at least 2, 3, 4, 5, 6, 7, 8, 9, or 10 nucleotides on or around the indicator nucleotides but comprising the complementary sequence of said indicator nucleotide as defined herein above in connection with SEQ ID NO: 1 to 14. In further embodiments, the molecules may preferably be complementary to at least 2, 3, 4, 5, 6, 7, 8, 9, or 10 nucleotides on or around the polymorphic site as defined herein above in connection with SEQ ID NO: 1 to 14, however comprising the wildtype sequence.

In preferred embodiments of the present invention said oligonucleotide as defined herein above may have a sequence complementary to a sequence including the indicator nucleotide of the SNPs of the present invention as defined herein above. In further embodiments the oligonucleotide may also have a complementary sequences towards the counter strand of said sequence including the indicator nucleotide of the SNPs of the present invention as defined herein above.

In further embodiments the present invention also relates to oligonucleotide molecules specifically binding in the vicinity of the polymorphic site as indicated herein above n the context of SEQ ID NO: 1 to 14. These oligonucleotides may be designed in the form of a pair of primers allowing the amplification of stretch of DNA, e.g. of a length of 50 bp, 75 bp, 100 bp, 150 bp, 200 bp, 250 bp, 300 bp, 400 bp, 500 bp, 750 bp, 1000 bp, or more around and including the polymorphic site of the SNPs of the present invention. Suitable sequence information may be derived from the sequence of SEQ ID NO: 1 to 14, the herein above indicated genomic sequence localization, which allows the skilled person to obtain the necessary context DNA sequence from data repositories, e.g. the human genome of build 37.1.

The term “probe specific for one or more polymorphic sites as defined herein above” as used herein refers piece of DNA, which is capable of specifically binding to a polymorphic site according to the present invention. The probe may, for example, be designed such that it only binds to a sequence comprising the indicator nucleotide, or the wildtype sequence, or a complementary strand thereof. In other embodiments the probe may be capable of binding to a polymorphic site according to the present invention, i.e. be able to bind to the wildtype sequence, the indicator nucleotide comprising sequence or any other variant at that position as defined herein above. The specificity of the probe may further be adjusted, for example in hybridization experiments, by the changing the concentration of salts, modifying the temperature of the reaction, adding further suitable compounds to the reaction etc. The probe may also be designed such that it binds outside of the polymorphic site, e.g. within the sequence of SEQ ID NO: 1 to 14, or a complementary sequence thereof.

The probe according to the present invention may, in further embodiments, be at least 90%, 91%, 92%, 93%, 94%, 95%, 96%, 97%, 98% or 99% or 99.5% or 99.6%, 99.7%, 99.8%, or 99.9% identical to the sequence of SEQ ID NO: 1 to 14, or to fragments thereof, which comprise the polymorphic site as defined herein above, wherein said sequence of SEQ ID NO: 1 to 14 comprises the respective indicator nucleotide as described herein above, or to any fragments of said sequences, or to the corresponding wildtype sequences as defined herein above, or to the complementary sequences of these sequences.

A probe according to the present invention may have any suitable length, e.g. a length of 15, 20, 30, 40, 50, 100, 150, 200, 300, 500, 1000 or more than 1000 nucleotides. The probe may further be suitable modified, e.g. by the addition of labels, e.g. fluorescent labels, dyes, radioactive labels etc.

In further embodiments, the probe may also be functionally adjusted to a detection method as described herein above.

In another aspect the present invention relates to the use of a nucleic acid molecule as defined herein above for detecting or diagnosing beta thalassemia in a subject. In preferred embodiment the present invention relates to the use of a nucleic acid molecule as defined herein above for detecting or diagnosing beta thalassemia minor in a subject. A nucleic acid molecule as defined herein above may be used as a template for a corresponding detection approach, e.g. based on the above defined methods. More preferably an affinity ligand for a polymorphic site according to the present invention, e.g. a oligonucleotide or probe, may be employed in a suitable method for the detection of the presence of a wildtype or indicator nucleotide at position 501 of SEQ ID NO: 1 to 14. Upon the determination of the corresponding sequence, the presence of beta thalassemia, preferably of beta thalassemia minor, may be confirmed or denied as described herein above.

In another particularly preferred embodiment the present invention relates to the use of a nucleic acid molecule as defined herein above for screening a population of subjects for the presence of beta thalassemia. The term “screening” refers to a detection program on a larger scale with detection/diagnosis facilities in big hospitals, and/or rural hospitals, and/or outpost stations throughout an entire region, state or nation. The screening may be performed according to standardized schemes as known to the person skilled in the art, e.g. based on the use of identical buffers solutions, nucleic acid molecules, labeling reagents etc. The results of the screening may be obtained locally or may be integrated in a regional, state- or nation-wide manner, e.g. in suitable databases, on the basis of corresponding platforms etc. In further embodiments, a screening may be carried out in a medical practice, e.g. region, state or nation-wide.

In further embodiments, the screening approach may be supplemented by a genetic counseling step, e.g. in case a beta thalassemia carrier is identified.

In a particularly preferred embodiment the screening may be carried out for beta thalassemia minor. In a particularly preferred embodiment the screening may be carried out for beta thalassemia carriers.

The term “population” as used herein refers to groups of similar subjects, e.g. people living in the same region, state or country, or being identified by other typical features of a genetic population. Particularly preferred are populations of subjects from South Asia. More preferred is an Indian population. Also envisaged are sub-populations, e.g. the sub-population of northern or southern Indian subjects.

In yet another aspect the present invention relates to a kit for detecting or diagnosing beta thalassemia in a subject, comprising an oligonucleotide specific for one or more polymorphic sites as defined herein above, or a probe specific for one or more polymorphic sites as defined herein above. In a particularly preferred embodiment said oligonucleotide has a sequence complementary to an indicator nucleotide as defined herein above. In another particularly preferred embodiment said beta thalassemia is beta thalassemia minor. In further embodiments the kit as defined herein above may comprise accessory ingredients such as PCR buffers, dNTPs, a polymerase, ions like bivalent cations or monovalent cations, hybridization solutions etc. In further embodiments the kit may also comprise accessory ingredients like secondary affinity ligands, e.g. secondary antibodies, detection dyes, or other suitable compound or liquids necessary for the performance of a nucleic acid detection. Such ingredients as well as further details would known to the person skilled in the art and may vary depending on the detection method carried out. Additionally, the kit may comprise an instruction leaflet and/or may provide information as to the relevance of the obtained results.

In yet another preferred embodiment the above mentioned method, composition, use, or kit relate to the assessment of the risk of developing beta thalassemia in a subject and/or in a subject's progeny. The term “assessment of the risk of developing beta thalassemia” as used herein refers to the person risk of a subject to develop during its lifetime a beta thalassemia phenotype. For example, if a subject is diagnosed to be afflicted by beta thalassemia according to the presently provided method, but shows no or only very moderate anemia, it may be assumed that there is a risk of developing a more severe form of an anemia during later decades of the life. This assumption may be associated with a suitable risk factor as would be known to the person skilled in the art.

The term “assessment of the risk of developing beta thalassemia in a subject's progeny” as used herein refers to the risk of developing beta thalassemia, e.g. beta thalassemia intermedia or beta thalassemia major, in the next generation, i.e. in a subject's child if a subject is diagnosed as beta thalassemia carrier. The risk may accordingly be calculated if a couple or a family presents itself, e.g. during a screening approach as defined herein above. Thus, if for both, man and woman, are diagnosed to be beta thalassemia carriers the risk that a child may develop beta thalassemia, and in particular the risk of developing a severe form of beta thalassemia, e.g. beta thalassemia intermedia or beta thalassemia major, may be considered as raised. The risk assessment may preferably be integrated in family planning or genetic counseling approaches, which may be offered in hospitals, specials medical practices or during medical campaigns.

The following examples and figures are provided for illustrative purposes. It is thus understood that the example and figures are not to be construed as limiting. The skilled person in the art will clearly be able to envisage further modifications of the principles laid out herein.

### EXAMPLES

### Example 1

### Identification of SNPs

For the identification of the SNPs the following experimental steps were carried out:

1. Samples were selected based on a number of parameters which includes:

a. Ethnicity—Samples were collected from the North Indian population

b. Sex

c. Age

d. Sample Type—Blood samples were collected from the individuals (both healthy and affected)

e. Family History

f. Medical History

A total of 161 samples were selected, with diseased samples being 71 and control samples being 90.

2. Clinicopathological information from patients and controls were collected with ‘Informed Consent’ which was approved in ‘Independent Ethical Committee’.

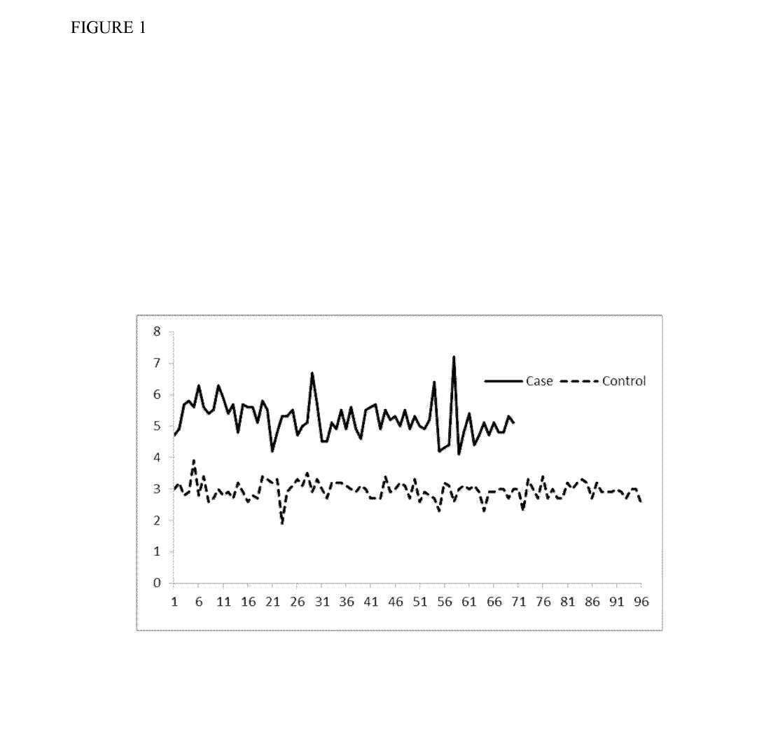

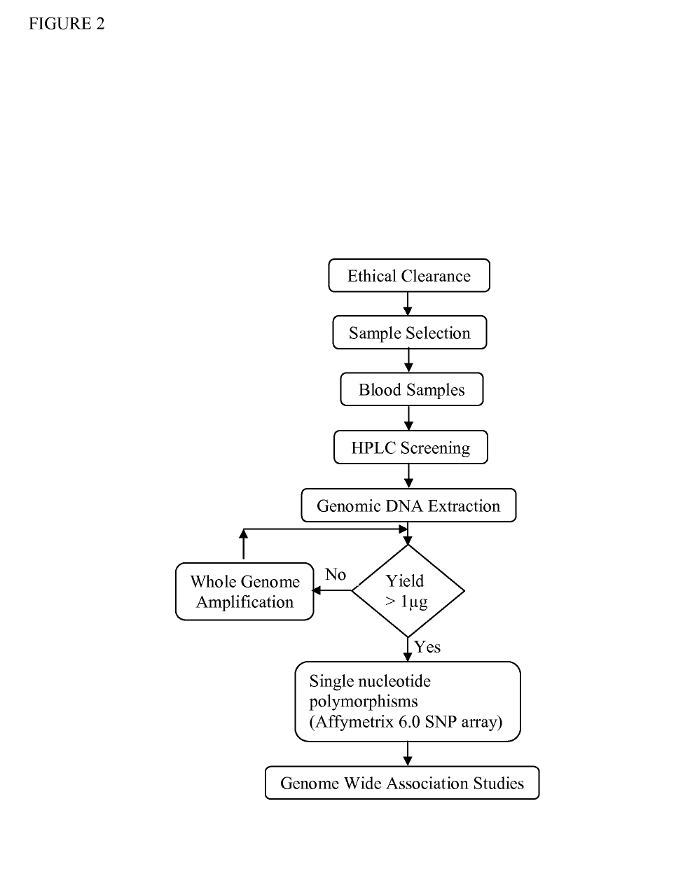

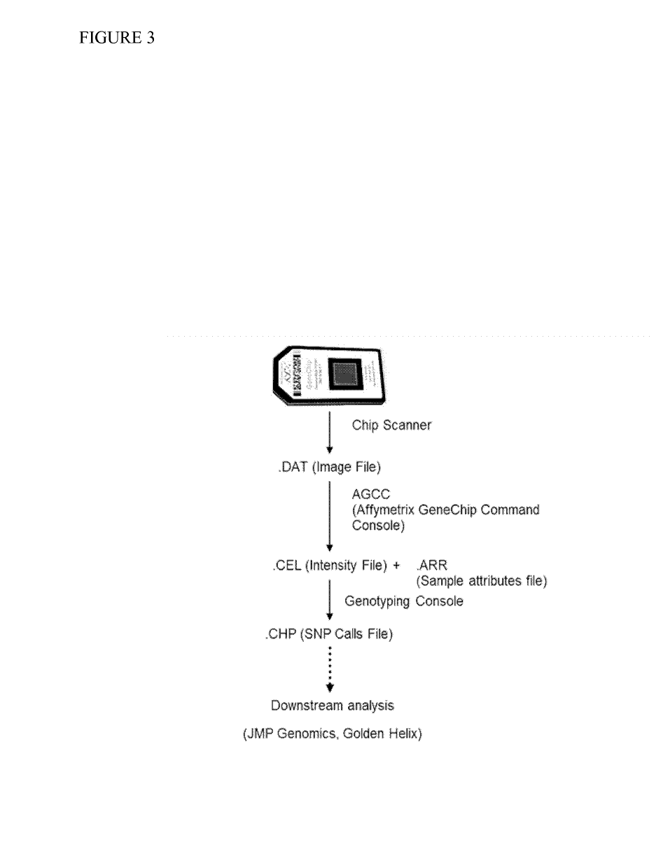

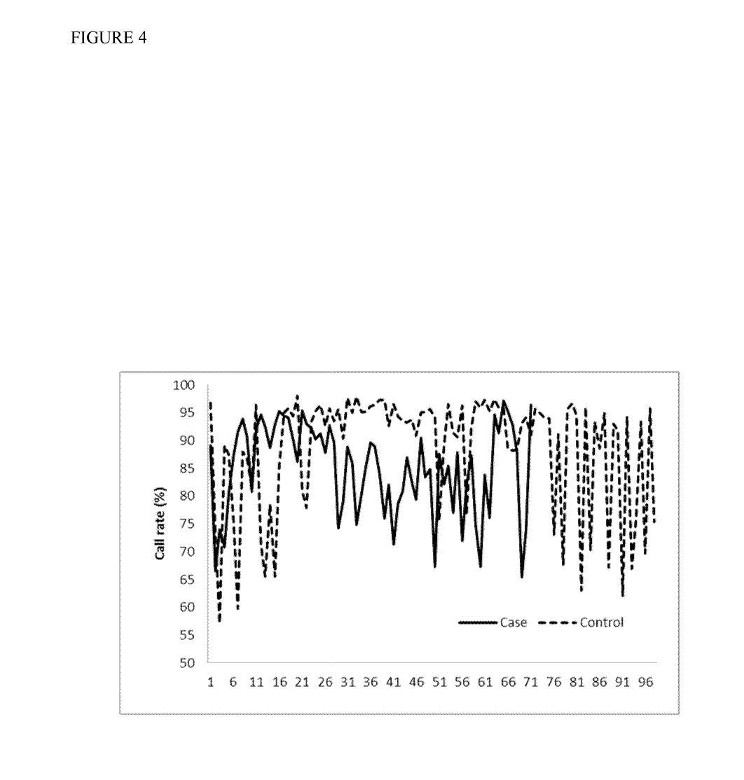

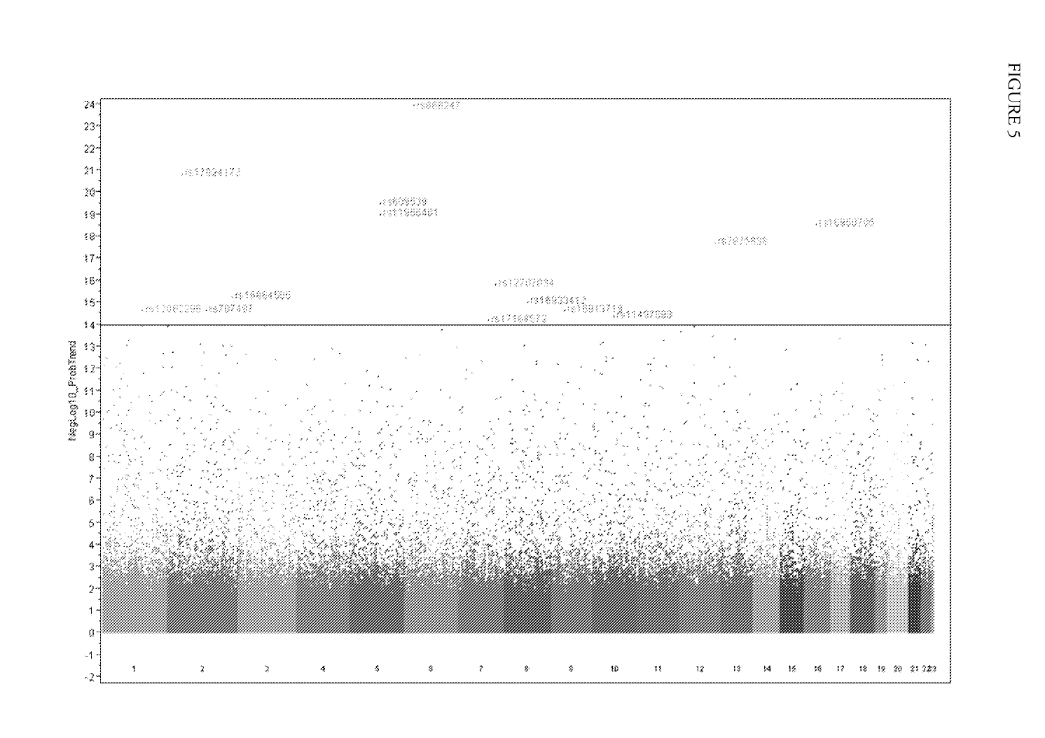

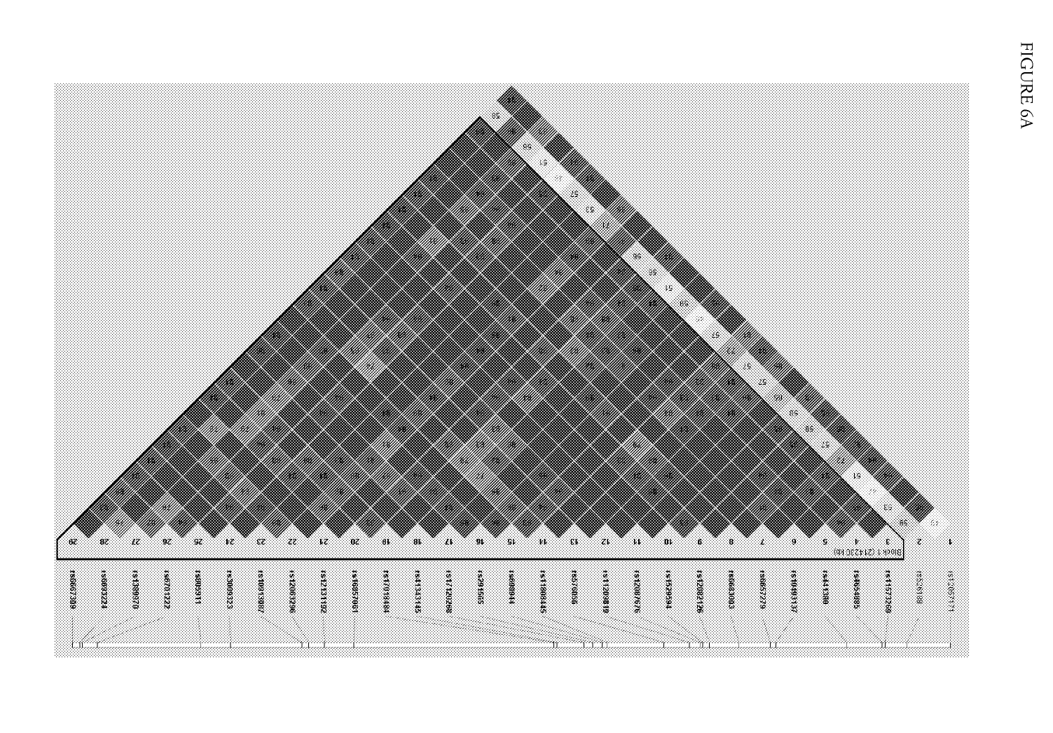

3. Blood samples were collected from the shortlisted individuals, screened for beta thalassemia trait using High Performance Liquid Chromatography (results are shown in FIG. 1) and DNA extraction and amplification was done using standard protocols as recommended by Affymetrix. An overall schema of the study is given in FIG. 2.

4. Using Affymetrix Genome Wide Human SNP Array 6.0, genotypes were generated on 906,000 SNPs in 71 individuals with beta thalassemia and in 90 controls. The steps involved in the data generation from SNP 6.0 Array include (standard protocol by Affymetrix):

a. Genomic DNA Plate Preparation: the concentration of human genomic DNA is quantified and accordingly, each sample is diluted to 50 ng/μl using reduced TE (Tris-Ethylenediamine tetra-acetic acid) buffer.

TABLE 1

| | | | | Con | Yield | Working Conc. |
|---|---|---|---|---|---|---|
| Reg. ID | 260 | 280 | 260/280 | ng/μL | (μg) | (ng/μl) |
| 166124 | 0.11 | 0.069 | 1.77 | 478 | 119.5 | 50 |
| 166125 | 0.133 | 0.83 | 1.78 | 576 | 144 | 50 |
| 166126 | 0.188 | 0.116 | 1.78 | 823 | 205.75 | 50 |
| 166128 | 0.087 | 0.057 | 1.77 | 349 | 87.25 | 50 |
| 166129 | 0.096 | 0.065 | 1.75 | 359 | 89.75 | 50 |
| 168083 | 0.262 | 0.171 | 1.75 | 1070 | 267.5 | 50 |
| 168085 | 0.412 | 0.251 | 1.79 | 1824 | 456 | 50 |
| 168087 | 0.366 | 0.229 | 1.77 | 1568 | 392 | 50 |
| 168088 | 0.295 | 0.184 | 1.77 | 1270 | 317.5 | 50 |

Table 1 shows the details of DNA extraction for certain samples (QC and final concentration

b. Sty restriction enzyme (RE) digestion: genomic DNA is digested with Sty1 restriction enzyme. A digestion master mix (dist. water, NE Buffer, Bovine Serum Albumin, Sty 1) is added to the genome and placed in thermocycler. The digest program essentially keeps the samples at 37° C. for 120 min and then 65° C. for 20 min.

c. Sty Ligation: digested samples are ligated with Sty ligator. The master mix consists of the ligase buffer, T4 DNA Ligase and Adaptor Sty I. The ligation mixture is kept at 16° C. for 180 min and then for 20 min at 70° C.

d. Sty PCR: PCR master mix consisting of primers, polymerase buffer, dNTPs and Taq DNA polymerase. The ligated samples are run through the PCR program in the PCR mix.

e. Nsp RE digestion: genomic samples are digested with Nsp I restriction enzyme using the same digestion protocol.

f. Nsp ligation: The Nsp I digested fragments are ligated with Nsp I adaptors using the same ligation protocol.

g. Nsp PCR: ligated fragments are run through another round of PCR.

h. PCR product pooling and purification: Sty I and Nsp I PCR products are pooled together into a single well plate. Beads are added to the mix and incubated. The pool is then transferred to a filter plate and vacuum-dried. The PCR products are washed and eluted out using an elution buffer.

i. Quantitation: DNA in each sample is quantified using a spectrophotometer.

j. Fragmentation: purified PCR products are fragmented using a fragmentation reagent and then assessed by gel electrophoresis.

k. Labeling: TdT enzyme is used to label the fragmented PCR products.

l. Target Hybridization: hybridization mix is added to each sample and the mix is denatured. After denaturation, the sample is loaded onto the SNP 6.0 microarray and incubated in the hybridization chamber for 16 to 18 hours.

m. After hybridization, the SNP array is washed properly to remove out non-specific binding and then scanned using GeneChip Scanner 3000 7G.

The software associated with the scanner will scan each and every spot on the chip, normalize the dye intensity values by performing background correction and produce a document with the raw and normalized intensity values for every probe on the array. Affymetrix GeneChip Command Console maps the pixel intensity to probe annotation (supplied by Affymetrix) to generate .CEL files that contain the signal values for the probe.

5. The .CEL files for each individual were subjected to quality control (QC). Some examples of various QCs performed on the samples are given in Table 2. Genotyping Console (GTC) was used to perform QC using the following metrics:

a. Contrast QC: A threshold of >=0.4 for each sample was set. Samples having contrast QC below this value were discarded. 48 cases and 66 controls had contrast QC values>=0.4 b. QC Call Rate: The threshold is set as 86%. All the 48 cases and 66 controls had QC call rate above 86%.

Typically, in good-quality data sets, 90 percent of samples should pass the QC Call Rate threshold and the average QC Call Rate should be in the mid-90 percent range. Occasionally, poor samples will pass the QC Call Rate metric, which is why Contrast QC is to be used for the SNP Array 6.0

TABLE 2

| | | | | QC Call | QC Call | |
|---|---|---|---|---|---|---|
| | | Contrast | QC Call | Rate | Rate | QC Call |
| File | Bounds | QC | Rate | (Nsp) | (Nsp/Sty) | Rate (Sty) |
| Philips_B65_(Genome | In | 1.03 | 95.7 | 96.14 | 96.61 | 92.75 |
| WideSNP_6).CEL | | | | | | |
| Philips_B67_(Genome | In | 0.96 | 94.04 | 93.19 | 95.69 | 90.82 |
| WideSNP_6).CEL | | | | | | |
| Philips_B129_(Genome | In | 0.77 | 95.76 | 93.83 | 97.6 | 93.4 |
| WideSNP_6).CEL | | | | | | |
| Philips_B35_(Genome | In | 1.81 | 94.67 | 93.7 | 95.93 | 92.59 |
| WideSNP_6).CEL | | | | | | |
| Philips_B122.CEL | Out | 0.33 | 92.89 | 95.37 | 93.47 | 88.24 |

Table 2 shows quality control of certain samples after processing of the arrays. Samples that are in-bounds were taken for further analysis.

6. The .CEL files of the samples are used to generate the genotype of the individual using GTC. As a result .CHP files are generated which contain the genotype of each SNP on the microarray for a particular individuals. The genotyped data was exported and this exported data was converted to pedigree format (.ped, .map and .info files) to facilitate analysis with HaploView.

7. Minor Allele Frequency (MAF) was obtained to filter irrelevant SNPs. All SNPs having MAF<0.05, non missing genotype rate≦0.9 and HWE p-value≦0.01 were excluded from further analysis.

8. Case-control association tests were carried out for finding out the association between markers and the trait using the case-control data. P-values from these tests are plotted along the marker map. The most significant p-values (>=10⁻¹⁴) were found at 14 polymorphic sites (Table 3 and 4).

9. Association found between a marker and a disease state was verified by subsequent independent study with different set of participants (Table 5)

TABLE 3

| | | | | Major | Wildtype | Assoc | |
|---|---|---|---|---|---|---|---|
| dbSNPrsID | Chromosome | Strand | Position | Allele | Allele | Allele | MAF |
| rs666247 | Chr06 | − | 20141438 | T | T | C | 0.21559633 |
| rs17024172 | Chr02 | + | 39785767 | A | A | G | 0.186363636 |
| rs609539 | Chr05 | − | 106932896 | A | G | A | 0.291262136 |
| rs11956461 | Chr05 | − | 104406522 | C | C | T | 0.160377358 |
| rs16950705 | Chr16 | − | 50619760 | C | C | T | 0.183962264 |
| rs7975838 | Chr12 | − | 115366107 | C | T | C | 0.327102804 |
| rs12707034 | Chr07 | + | 131667698 | T | T | C | 0.233009709 |
| rs16864505 | Chr02 | + | 223727014 | C | C | T | 0.199029126 |
| rs16933412 | Chr08 | + | 68660459 | T | T | C | 0.169724771 |
| rs12063296 | Chr01 | + | 172196522 | A | A | G | 0.132075472 |
| rs707497 | Chr02 | − | 124781779 | T | T | C | 0.223300971 |
| rs16913719 | Chr09 | − | 28809674 | C | C | T | 0.14159292 |
| rs11497898 | Chr10 | − | 66188858 | T | T | C | 0.135514019 |
| rs17168572 | Chr07 | − | 96903955 | A | A | G | 0.133027523 |

Table 3 shows short-listed SNPs that showed significant association

TABLE 4

| | | | Holm | | | Benjamini & | Benjamini & |
|---|---|---|---|---|---|---|---|
| | | Bonferroni | (1979) | Sidak | Sidak | Hochberg | Yekutieli |
| | | single- | step- | single- | step- | (1995) | (2001) |
| | Unadjusted | step | down | step | down | step-up | step-up |
| | p- | adjusted | adjusted | adjusted | adjusted | FDR | FDR |
| dbSNPrsID | value | p-values | p-values | p-values | p-values | control | control |
| rs666247 | 1.32E−19 | 1.85E−18 | 1.85E−18 | INF | INF | 1.85E−18 | 6.00E−18 |
| rs12707034 | 2.68E−19 | 3.75E−18 | 3.48E−18 | INF | INF | 1.88E−18 | 6.10E−18 |
| rs707497 | 2.40E−18 | 3.35E−17 | 2.87E−17 | INF | INF | 1.12E−17 | 3.63E−17 |
| rs17024172 | 5.61E−17 | 7.86E−16 | 6.17E−16 | 1.55E−15 | 1.22E−15 | 1.96E−16 | 6.39E−16 |
| rs16950705 | 2.28E−16 | 3.19E−15 | 2.28E−15 | 3.11E−15 | 2.22E−15 | 6.39E−16 | 2.08E−15 |
| rs11956461 | 2.98E−16 | 4.17E−15 | 2.68E−15 | 4.66E−15 | 3.00E−15 | 6.96E−16 | 2.26E−15 |
| rs609539 | 4.61E−16 | 6.45E−15 | 3.69E−15 | 6.22E−15 | 3.55E−15 | 9.22E−16 | 3.00E−15 |
| rs7975838 | 1.59E−14 | 2.23E−13 | 1.12E−13 | 2.24E−13 | 1.12E−13 | 2.79E−14 | 9.07E−14 |
| rs12063296 | 2.97E−13 | 4.15E−12 | 1.78E−12 | 4.15E−12 | 1.78E−12 | 4.61E−13 | 1.50E−12 |
| rs16913719 | 4.65E−13 | 6.51E−12 | 2.32E−12 | 6.51E−12 | 2.32E−12 | 6.51E−13 | 2.12E−12 |
| rs11497898 | 5.79E−13 | 8.11E−12 | 2.32E−12 | 8.11E−12 | 2.32E−12 | 7.37E−13 | 2.40E−12 |
| rs17168572 | 7.76E−13 | 1.09E−11 | 2.33E−12 | 1.09E−11 | 2.33E−12 | 8.50E−13 | 2.77E−12 |
| rs16933412 | 7.90E−13 | 1.11E−11 | 2.33E−12 | 1.11E−11 | 2.33E−12 | 8.50E−13 | 2.77E−12 |
| rs16864505 | 2.29E−12 | 3.21E−11 | 2.33E−12 | 3.21E−11 | 2.33E−12 | 2.29E−12 | 7.44E−12 |

Table 4 depicts multiple hypothesis testing correction of observed p-values of most significant SNPs.

TABLE 5

| dbSNP_RS_ID | Strand | Associated Gene |
|---|---|---|
| rs11497898 | − | NR_001562 // upstream // 66427 // --- // ANXA2P1 // 303 // annexin A2 |
| | | pseudogene 1 /// NM_000972 // upstream // 855827 // Hs.499839 // RPL7A |
| | | // 6130 // ribosomal protein L7a /// ENST00000356292 // upstream // |
| | | 66421 // Hs.511605 // ANXA2 // 302 // annexin A2 /// ENST00000323345 |
| | | // upstream // 855827 // Hs.499839 // RPL7A // 6130 // ribosomal protein |
| | | L7a |
| rs11956461 | − | NR_000039 // upstream // 56551 // --- // RAB9P1 // 9366 // RAB9, member |
| | | RAS oncogene family, pseudogene 1 /// NM_031438 // upstream // |
| | | 1480133 // Hs.434289 // NUDT12 // 83594 // nudix (nucleoside |
| | | diphosphate linked moiety X)-type motif 12 /// ENST00000333274 // |
| | | downstream // 2337727 // Hs.288741 // EFNA5 // 1946 // ephrin-A5 /// |
| | | ENST00000230792 // upstream // 1480133 // Hs.434289 // NUDT12 // |
| | | 83594 // nudix (nucleoside diphosphate linked moiety X)-type motif 12 |
| rs12063296 | + | NM_172071 // intron // 0 // Hs.30258 // RC3H1 // 149041 // ring finger |
| | | and CCCH-type zinc finger domains 1 /// ENST00000258349 // intron // 0 |
| | | // Hs.30258 // RC3H1 // 149041 // ring finger and CCCH-type zinc finger |
| | | domains 1 /// ENST00000367696 // intron // 0 // Hs.30258 // RC3H1 // |
| | | 149041 // ring finger and CCCH-type zinc finger domains 1 |
| rs12707034 | + | NM_020911 // intron // 0 // Hs.511454 // PLXNA4 // 91584 // plexin A4 /// |
| | | ENST00000408969 // intron // 0 // Hs.511454 // PIJCNA4 // 91584 // plexin |
| | | A4 |
| rs16864505 | + | NM_003469 // downstream // 442889 // Hs.516726 // SCG2 // 7857 // |
| | | secretogranin II (chromogranin C) /// NM_080671 // downstream // 98417 |
| | | // Hs.348522 // KCNE4 // 23704 // potassium voltage-gated channel, Isk- |
| | | related family, member 4 /// ENST00000305409 // downstream // 442891 // |
| | | Hs.516726 // SCG2 // 7857 // secretogranin II (chromogranin C) /// |
| | | ENST00000281830 // downstream // 98417 // Hs.348522 // KCNE4 // |
| | | 23704 //potassium voltage-gated channel, Isk-related family, member 4 |
| rs16913719 | − | NM_002396 // downstream // 1004395 // Hs.233119 // ME2 // 4200 // |
| | | malic enzyme 2, NAD(+)-dependent, mitochondrial /// NM_152570 // |
| | | upstream // 149394 // Hs.715650 // LINGo2 // 158038 // leucine rich |
| | | repeat and Ig domain containing 2 /// ENST00000321341 // downstream // |
| | | 1004395 // Hs.233119 // ME2 // 4200 // malic enzyme 2, NAD(+)- |
| | | dependent, mitochondrial /// ENST00000379992 // upstream // 149391 // |
| | | Hs.650389 // LINGO2 // 158038 // leucine rich repeat and Ig domain |
| | | containing 2 |
| rs16933412 | + | NM_020361 // intron // 0 // Hs.658850 // CPA6 // 57094 // |
| | | carboxypeptidase A6 /// NM_001127445 // intron // 0 // Hs.658850 // |
| | | CPA6 // 57094 // carboxypeptidase A6 /// ENST00000297769 // intron // 0 |
| | | // Hs.658850 // CPA6 // 57094 // carboxypeptidase A6 /// |
| | | ENST00000297770 // intron // 0 // Hs.658850 // CPA6 // 57094 // |
| | | carboxypeptidase A6 |
| rs16950705 | − | NM_001146188 // downstream // 409658 // --- // TOX3 // 27324 // TOX |
| | | high mobility group box family member 3 /// NR_002944 // downstream // |
| | | 381232 // --- // HNRPA1L-2 // 664709 // heterogeneous nuclear |
| | | ribonucleoprotein A1 pseudogene /// ENST00000219746 // downstream // |
| | | 409665 // Hs.460789 // TOX3 // 27324 // TOX high mobility group box |
| | | family member 3 /// ENST00000357495 // downstream // 381241 // |
| | | Hs.447506 // HNRNPA1L2 // 144983 // heterogeneous nuclear |
| | | ribonucleoprotein A1-like 2 |
| rs17024172 | + | NM_152390 // intron // 0 // Hs.40808 // TMEM178 // 130733 // |
| | | transmembrane protein 178 /// ENST00000281961 // intron // 0 // |
| | | Hs.40808 // TMEM178 // 130733 // transmembrane protein 178 |
| rs17168572 | − | NM_013998 // upstream // 295251 // Hs.2563 // TAC1 // 6863 // |
| | | tachykinin, precursor 1 /// NM_020186 // downstream // 254946 // |
| | | Hs.592269 // ACN9 // 57001 // ACN9 homolog (S. cerevisiae) /// |
| | | ENST00000346867 // upstream // 295355 // Hs.2563 // TAC1 // 6863 // |
| | | tachykinin, precursor 1 /// ENST00000360382 // downstream // 254948 // |
| | | Hs.592269 // ACN9 // 57001 // ACN9 homolog (S. cerevisiae) |
| rs609539 | − | NM_001962 // intron // 0 // Hs.288741 // EFNA5 // 1946 // ephrin-A5 /// |
| | | ENST00000333274 // intron // 0 // Hs.288741 // EFNA5 // 1946 // ephrin- |
| | | A5 |
| rs666247 | − | NM_001080480 // downstream // 67475 // Hs.377830 // MBOAT1 // |
| | | 154141 // membrane bound O-acyltransferase domain containing 1 /// |
| | | NM_001546 // downstream // 192545 // Hs.519601 // ID4 // 3400 // |
| | | inhibitor of DNA binding 4, dominant negative helix-loop-helix protein /// |
| | | ENST00000324607 // downstream // 67475 // Hs.377830 // MBOAT1 // |
| | | 154141 // membrane bound O-acyltransferase domain containing 1 /// |
| | | ENST00000378700 // downstream // 192545 // Hs.519601 // ID4 // 3400 // |
| | | inhibitor of DNA binding 4, dominant negative helix-loop-helix protein |
| rs707497 | − | NM_130773 // intron // 0 // Hs.660653 // CNTNAP5 // 129684 // contactin |
| | | associated protein-like 5 /// ENST00000285362 // intron // 0 // Hs.660653 |
| | | // CNTNAP5 // 129684 // contactin associated protein-like 5 |
| rs7975838 | − | NR_027345 // upstream // 89502 // --- // NCRNA00173 // 100287569 // |
| | | non protein coding RNA 173 /// NM_015335 // upstream // 166733 // |
| | | Hs.603766 // MED13L // 23389 // mediator complex subunit 13-like /// |
| | | ENST00000306985 //upstream // 115461 // Hs.506947 // MAP1LC3B2 // |
| | | 643246 // microtubule-associated protein 1 light chain 3 beta 2 /// |
| | | ENST00000281928 // upstream // 166581 // Hs.603766 // MED13L // |
| | | 23389 // mediator complex subunit 13-like |

Table 5 shows shortlisted SNPs according to the present invention and associated genes.

### Example 2

### Estimation of Linkage Disequilibrium

Associated SNPs were extracted for the chromosomes on which 14 most significant SNPs had been observed using a lower threshold (chi-square p-value≦10⁻¹⁰) and linkage disequilibria among them were estimated. SNPs with high LD between them were visualized using Haploview and are shown in FIG. 6 (highest LD is between SNPs showing dark black blocks: logarithm of odds≧2, D′=1). Strength of association between these haplotype blocks thus found and the affected status were estimated and shown in Table 6. When designing an array for checking disease status, these haplotype blocks showing association with a significant p-value (≦10⁻¹⁰) may be included in that. All SNPs captured into those haplotype were included in the test; the presence and analysis of a significant haplotype in a subject/patient genotype is assumed to be helpful in the diagnostic process.

TABLE 6

| | | Case, Control Ratio | Case, Control | | |
|---|---|---|---|---|---|
| Block | Freq | Counts | freq | Chi Square | p-Value |
| Chr1 | 0.428 | 43.2:43.8, 51.0:80.5 | 0.496, 0.388 | 2.509 | 0.1132 |
| Block1 | | | | | |
| | 0.248 | 1.0:86.0, 53.5:78.0 | 0.011, 0.407 | 43.81 | 3.62E−11 |
| | 0.081 | 17.8:69.2, 0.0:131.5 | 0.204, 0 | 29.263 | 6.32E−08 |
| | 0.053 | 2.5:84.5, 9.1:122.3 | 0.028, 0.07 | 1.781 | 0.182 |
| | 0.048 | 4.1:82.9, 6.5:125.0 | 0.047, 0.049 | 0.004 | 9.48E−01 |
| | 0.023 | 2.2:84.8, 3.0:128.5 | 0.025, 0.023 | 0.007 | 0.9327 |
| | 0.016 | 1.9:85.1, 1.7:129.8 | 0.021, 0.013 | 0.256 | 0.6131 |
| | 0.014 | 3.2:83.8, 0.0:131.5 | 0.036, 0 | 4.831 | 0.0279 |
| Chr2 | 0.83 | 60.7:33.3, 126.9:5.1 | 0.646, 0.961 | 38.654 | 5.06E−10 |
| Block1 | | | | | |
| | 0.105 | 23.7:70.3, 0.0:132.0 | 0.252, 0 | 37.19 | 1.07E−09 |
| | 0.014 | 2.1:91.9, 1.1:130.9 | 0.022, 0.008 | 0.773 | 0.3794 |
| | 0.013 | 2.0:92.0, 1.0:131.0 | 0.021, 0.008 | 0.803 | 0.3701 |
| Chr2 | 0.745 | 47.5:42.5, 116.4:13.0 | 0.528, 0.9 | 38.929 | 4.40E−10 |
| Block2 | | | | | |
| | 0.095 | 20.8:69.2, 0.0:129.3 | 0.231, 0 | 33.041 | 9.02E−09 |
| | 0.057 | 7.0:83.0, 5.5:123.9 | 0.078, 0.042 | 1.243 | 0.265 |
| | 0.025 | 0.6:89.4, 5.0:124.3 | 0.006, 0.039 | 2.298 | 0.1295 |
| | 0.019 | 2.1:87.9, 2.0:127.3 | 0.024, 0.016 | 0.182 | 0.6698 |
| Chr5 | 0.811 | 59.1:36.9, 125.8:6.2 | 0.615, 0.953 | 41.269 | 1.33E−10 |
| Block1 | | | | | |
| | 0.085 | 19.3:76.7, 0.0:132.0 | 0.201, 0 | 29.049 | 7.06E−08 |
| | 0.043 | 5.8:90.2, 4.1:127.9 | 0.06, 0.031 | 1.162 | 0.281 |
| | 0.015 | 3.4:92.6, 0.0:132.0 | 0.035, 0 | 4.714 | 0.0299 |
| Chr6 | 0.793 | 47.7:46.3, 131.5:0.5 | 0.507, 0.996 | 80.024 | 3.70E−19 |
| Block1 | | | | | |
| | 0.11 | 24.9:69.1, 0.0:132.0 | 0.265, 0 | 39.325 | 3.59E−10 |
| | 0.07 | 15.3:78.7, 0.5:131.5 | 0.163, 0.004 | 21.421 | 3.69E−06 |
| Chr7 | 0.795 | 55.3:40.7, 126.0:6.0 | 0.576, 0.954 | 48.797 | 2.84E−12 |
| Block1 | | | | | |
| | 0.105 | 23.9:72.1, 0.0:132.0 | 0.249, 0 | 36.689 | 1.39E−09 |
| | 0.045 | 6.3:89.7, 4.0:128.0 | 0.066, 0.03 | 1.627 | 0.2022 |
| | 0.012 | 2.8:93.2, 0.0:132.0 | 0.029, 0 | 3.857 | 0.0495 |
| Chr8 | 0.787 | 54.3:37.7, 121.9:10.1 | 0.59, 0.924 | 35.872 | 2.11E−09 |
| Block1 | 0.107 | 23.9:68.1, 0.0:132.0 | 0.259, 0 | 38.334 | 5.96E−10 |
| | 0.034 | 3.6:88.4, 4.0:128.0 | 0.039, 0.03 | 0.13 | 0.7185 |
| | 0.021 | 1.8:90.2, 3.0:129.0 | 0.019, 0.023 | 0.031 | 0.8603 |
| | 0.014 | 3.1:88.9, 0.0:132.0 | 0.033, 0 | 4.443 | 0.035 |
| Chr10 | 0.818 | 57.7:38.3, 128.8:3.2 | 0.601, 0.976 | 52.395 | 4.54E−13 |
| Block1 | | | | | |
| | 0.104 | 23.8:72.2, 0.0:132.0 | 0.248, 0 | 36.576 | 1.47E−09 |
| | 0.016 | 2.4:93.6, 1.1:130.9 | 0.025, 0.009 | 0.995 | 0.3185 |
| | 0.013 | 3.1:92.9, 0.0:132.0 | 0.032, 0 | 4.283 | 0.0385 |
| Chr12 | 0.821 | 60.7:35.3, 126.6:5.4 | 0.632, 0.959 | 40.617 | 1.85E−10 |
| Block1 | | | | | |
| | 0.109 | 24.9:71.1, 0.0:132.0 | 0.26, 0 | 38.475 | 5.55E−10 |
| | 0.039 | 4.6:91.4, 4.4:127.6 | 0.048, 0.033 | 0.336 | 0.5621 |
| | 0.012 | 2.7:93.3, 0.0:132.0 | 0.028, 0 | 3.751 | 0.0528 |

Table 6 shows the results of the haplotype analysis

1. An isolated nucleic acid molecule selected from the group comprising:
(i) SEQ ID NO: 1 [rs666247] except for a single polymorphic change at position 501, where wildtype nucleotide T is replaced by indicator nucleotide C;
(ii) SEQ ID NO: 2 [rs12707034] except for a single polymorphic change at position 501, where wildtype nucleotide T is replaced by indicator nucleotide C;
(iii) SEQ ID NO: 3 [rs707497] except for a single polymorphic change at position 501, where wildtype nucleotide T is replaced by indicator nucleotide C;
(iv) SEQ ID NO: 4 [rs17024172] except for a single polymorphic change at position 501, where wildtype nucleotide A is replaced by indicator nucleotide G;
(v) SEQ ID NO: 5 [rs16950705] except for a single polymorphic change at position 501, where wildtype nucleotide C is replaced by indicator nucleotide T;
(vi) SEQ ID NO: 6 [rs11956461] except for a single polymorphic change at position 501, where wildtype nucleotide C is replaced by indicator nucleotide T;
(vii) SEQ ID NO: 7 [rs609539] except for a single polymorphic change at position 501, where wildtype nucleotide G is replaced by indicator nucleotide A;
(viii) SEQ ID NO: 8 [rs7975838] except for a single polymorphic change at position 501, where wildtype nucleotide T is replaced by indicator nucleotide C;
(ix) SEQ ID NO: 9 [rs12063296] except for a single polymorphic change at position 501, where wildtype nucleotide A is replaced by indicator nucleotide G;
(x) SEQ ID NO: 10 [rs16913719] except for a single polymorphic change at position 501, where wildtype nucleotide C is replaced by indicator nucleotide T;
(xi) SEQ ID NO: 11 [rs11497898] except for a single polymorphic change at position 501, where wildtype nucleotide T is replaced by indicator nucleotide C;
(xii) SEQ ID NO: 12 [rs17168572] except for a single polymorphic change at position 501, where wildtype nucleotide A is replaced by indicator nucleotide G;
(xiii) SEQ ID NO: 13 [rs16933412] except for a single polymorphic change at position 501, where wildtype nucleotide T is replaced by indicator nucleotide C; and
(xiv) SEQ ID NO: 14 [rs16864505] except for a single polymorphic change at position 501, where wildtype nucleotide C is replaced by indicator nucleotide T.

2. The isolated nucleic acid of claim 1 or a group of nucleic acids of claim 1, wherein a panel of at least 1, 2, 3, 4, 5, 6, 7, 8, 9, 10, 11, 12, 13, or all of said polymorphic, changed sequences comprising said indicator nucleotides constitutes a marker for beta thalassemia, preferably of beta thalassemia minor.

3. The isolated nucleic acid or group of nucleic acids of claim 2, wherein said panel comprises at least:
(i) SEQ ID NO: 1 except for a single polymorphic change at position 501, where wildtype nucleotide T is replaced by indicator nucleotide C; or
(ii) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; or
(iii) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; or
(iv) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; or
(v) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 5 except for a single polymorphic change at position 501, where wildtype nucleotide C is replaced by indicator nucleotide T; or
(vi) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 5 except for a single polymorphic change at position 501, where wildtype nucleotide C is replaced by indicator nucleotide T; and SEQ ID NO: 6 except for a single polymorphic change at position 501, where wildtype nucleotide C is replaced by indicator nucleotide T; or
(vii) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 5 except for a single polymorphic change at position 501, where wildtype nucleotide C is replaced by indicator nucleotide T; and SEQ ID NO: 6 except for a single polymorphic change at position 501, where wildtype nucleotide C is replaced by indicator nucleotide T; and SEQ ID NO: 7 except for a single polymorphic change at position 501, where wildtype nucleotide G is replaced by indicator nucleotide A; or
(viii) SEQ ID NO: 1 except for a single polymorphic change at position 501, where wildtype nucleotide T is replaced by indicator nucleotide C; and SEQ ID NO: 2 except for a single polymorphic change at position 501, where wildtype nucleotide T is replaced by indicator nucleotide C; and SEQ ID NO: 3 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 5 except for a single polymorphic change at position 501, where wildtype nucleotide C is replaced by indicator nucleotide T; and SEQ ID NO: 6 except for a single polymorphic change at position 501, where wildtype nucleotide C is replaced by indicator nucleotide T; and SEQ ID NO: 7 except for a single polymorphic change at position 501, where wildtype nucleotide G is replaced by indicator nucleotide A; and SEQ ID NO: 8 except for a single polymorphic change at position 501, where wildtype nucleotide T is replaced by indicator nucleotide C; and SEQ ID NO: 9 except for a single polymorphic change at position 501, where wildtype nucleotide A is replaced by indicator nucleotide G; and SEQ ID NO: 10 except for a single polymorphic change at position 501, where wildtype nucleotide C is replaced by indicator nucleotide T; and SEQ ID NO: 11 except for a single polymorphic change at position 501, where wildtype nucleotide T is replaced by indicator nucleotide C; and SEQ ID NO: 12 except for a single polymorphic change at position 501, where wildtype nucleotide A is replaced by indicator nucleotide G; and SEQ ID NO: 13 except for a single polymorphic change at position 501, where wildtype nucleotide T is replaced by indicator nucleotide C; and SEQ ID NO: 14 except for a single polymorphic change at position 501, where wildtype nucleotide C is replaced by indicator nucleotide T; or
(ix) SEQ ID NO: 8 except for a single polymorphic change at position 501, where wildtype nucleotide T is replaced by indicator nucleotide C; and SEQ ID NO: 14 except for a single polymorphic change at position 501, where wildtype nucleotide C is replaced by indicator nucleotide T; or
(x) SEQ ID NO: 8 except for a single polymorphic change at position 501, where wildtype nucleotide T is replaced by indicator nucleotide C; and SEQ ID NO: 9 except for a single polymorphic change at position 501, where wildtype nucleotide A is replaced by indicator nucleotide G; or
(xi) SEQ ID NO: 2 except for a single polymorphic change at position 501, where wildtype nucleotide T is replaced by indicator nucleotide C; and SEQ ID NO: 4 except for a single polymorphic change at position 501, where wildtype nucleotide A is replaced by indicator nucleotide G; and SEQ ID NO: 13 except for a single polymorphic change at position 501, where wildtype nucleotide T is replaced by indicator nucleotide C.

4. Method for detecting or diagnosing beta thalassemia, preferably of beta thalassemia minor, in a subject comprising the steps of:
(a) isolating a nucleic acid from a subject's sample
(b) determining the nucleotide sequence and/or molecular structure present at one or more polymorphic sites as defined in claim 1;
wherein the presence of an indicator nucleotide is indicative of the presence of beta thalassemia.

5. The method of claim 4, wherein said determination of the nucleotide sequence is carried out through allele-specific oligonucleotide (ASO)-dot blot analysis, primer extension assays, iPLEX SNP genotyping, Dynamic allele-specific hybridization (DASH) genotyping, the use of molecular beacons, tetra primer ARMS PCR, a flap endonuclease invader assay, an oligonucleotide ligase assay, PCR-single strand conformation polymorphism (SSCP) analysis, quantitative real-time PCR assay, SNP microarray based analysis, restriction enzyme fragment length polymorphism (RFLP) analysis, targeted resequencing analysis and/or whole genome sequencing analysis.

6. The method of claim 4, wherein the method comprises as additional step the determination of the Hb A2 concentration in the sample.

7. The method of claim 6, wherein said determination of Hb A2 concentration is carried out via HPLC, microchromatography, isoelectric focusing, or capillary electrophoresis.

8. The method of claim 4, wherein said sample is a mixture of tissues, organs, cells and/or fragments thereof, or a tissue or organ specific sample, such as a tissue biopsy from vaginal tissue, tongue, pancreas, liver, spleen, ovary, muscle, joint tissue, neural tissue, gastrointestinal tissue, tumor tissue, or a body fluid, blood, serum, saliva, or urine, preferably blood.

9. The method of claim 4, comprising the determination of the nucleotide sequence and/or molecular structure present at polymorphic sites of SEQ ID NO: 8 and SEQ ID NO: 9 and the detection of a DNAse hypersensitivity site in the genomic vicinity of SEQ ID NO: 8 and/or SEQ ID NO: 9, wherein the presence of an indicator nucleotide as defined in any one of claims 1 to 3 and the presence of said DNAse hypersensitivity site is indicative of the presence of beta thalassemia.

10. The method of claim 4, comprising the determination of the nucleotide sequence and/or molecular structure present at polymorphic sites of SEQ ID NO: 2, SEQ ID NO: 4 and SEQ ID NO: 13 and the detection of a histone 3 lysine 27 trimethylation in the genomic vicinity of SEQ ID NO: 2 and/or SEQ ID NO: 4 and/or SEQ ID NO: 13, wherein the presence of an indicator nucleotide and the presence of said histone 3 lysine 27 trimethylation is indicative of the presence of beta thalassemia.

11. (canceled)

12. (canceled)

13. Use of a nucleic acid molecule as defined in claim 1 for detecting or diagnosing beta thalassemia, preferably of beta thalassemia minor, in a subject, or for screening a population of subjects, preferably an South Asian population of subjects, for the presence of beta thalassemia, preferably of beta thalassemia minor.

14. (canceled)

15. The method of claim 1, wherein said diagnosis of beta thalassemia comprises assessing the risk of developing beta thalassemia in a subject and/or in a subject's progeny.
